(19) United States
(12) Patent Application Publication
(10) Pub. No.: US 2007/0036775 A1
(43) Pub. Date: Feb. 15, 2007

(54) Method of modulating or examining Ku70 levels in cells

(76) Inventors: Shigemi Matsuyama, Glendale, WI (US); Weiyong Sun, Kawasaki City (JP)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 11/580,478

(22) Filed: Oct. 13, 2006

Related U.S. Application Data
(62) Division of application No. 10/924,060, filed on Aug. 23, 2004, now abandoned.
Application No. 10/924,060 is a continuation of application No. 10/247,045, filed on Sep. 19, 2002, now abandoned.
(60) Provisional application No. 60/324,292, filed on Sep. 24, 2001.
(60) Provisional application No. 60/378,585, filed on May 8, 2002.
(60) Provisional application No. 60/364,287, filed on Mar. 14, 2002.

(51) Int. Cl. A61K 48/00; C12Q 1/68

(52) U.S. Cl. 424/93.21; 435/6

(57) ABSTRACT

A method of predicting whether cells would respond to therapies which are mediated through Bax-regulated apoptosis is disclosed. In one embodiment, the method comprises the step of: (a) examining the intensity of the expression of the Bax protein or mRNA in a cell relative to a control, and (b) based on that intensity level, predicting whether cells will respond to therapies which are mediated through Bax-regulated apoptosis, wherein a high Bax level indicates that one may lower Ku70 levels and increase sensitivity to apoptosis. In another embodiment, the invention is a method of sensitizing cells to cancer therapy, comprising the step of reducing the cell's native Ku70 protein level. In another embodiment the invention is method of treating cell death-related diseases comprising the step of increasing cellular Ku70 protein level.

### CROSS-REFERENCE TO RELATED APPLICATION

This application claims priority from U.S. provisional 60/324,292, filed Sep. 24, 2001; U.S. provisional 60/378,585, filed May 8, 2002 and U.S. provisional 60/364,287, filed Mar. 14, 2002. These provisional applications are incorporated by reference herein.

### STATEMENT REGARDING FEDERALLY SPONSORED RESEARCH OR DEVELOPMENT

- - -

### BACKGROUND OF THE INVENTION

Bcl-2 family proteins are known to regulate a distal step in an evolutionarily conserved pathway for programmed cell death and apoptosis, with some members functioning as suppressors of apoptosis and others as promoters of cell death (Gross, et al., 1999; Reed, 1997b). In mammalian cells, Bcl-2 family proteins are known to control mitochondria-dependent cell death cascades (Adams and Cory, 1998; Green and Reed, 1998; Reed, et al., 1998). Mitochondria release apoptogenic factors during apoptosis such as Cytochrome c, apoptosis-inducing factor (AIF), and SMAC/DIABLO (Green, 2000). Cytochrome c released from mitochondria into the cytosol space triggers Apaf-1-dependent caspase activation leading cells to death (Green, 2000; Zou, et al., 1997). Pro-apoptotic Bcl-2 family proteins such as Bax promote Cytochrome c release from mitochondria (Jurgensmeier, et al., 1998). On the other hand, anti-apoptotic Bcl-2 family proteins such as Bcl-2 suppress Cytochrome c release from mitochondria, thereby protecting cells from apoptotic signals triggered by several stimuli (Kluck, et al., 1997; Yang, et al., 1997). The relative ratios of these various pro- and anti-apoptotic members of the Bcl-2 family have been known to determine the sensitivity of cells to diverse apoptotic stimuli (Oltvai and Korsmeyer, 1994) including chemotherapeutic drugs and radiation, growth factor deprivation, loss of cell attachment to extracellular matrix, hypoxia (a common occurrence in the centers of large tumors), and lysis by cytotoxic T-cells (Adams and Cory, 1998; Green and Reed, 1998; Gross, et al., 1999; Reed, 1997a).

Among pro-apoptotic Bcl-2 family members, Bax and Bak play a key role for apoptosis induction. The double knock out of these genes in mice resulted in the resistance of the cells to several cell death stimuli known to trigger mitochondria-dependent apoptosis, such as UV-irradiation, staurosporin (pan-kinase inhibitor), and some anti-cancer drugs (Wei, et al., 2001). Bax normally resides in the cytosol in a quiescent state. Upon receipt of apoptotic stimuli, Bax translocates into mitochondria (Wolter, et al., 1997), and promotes Cytochrome c release, possibly by forming a pore in the mitochondrial outer membrane (Korsmeyer, et al., 2000; Saito, et al., 2000). On the other hand, anti-apoptotic family proteins such as Bcl-2 and Bcl-XL reside in the mitochondrial membrane and antagonize the cytotoxic activity of Bax moved from the cytosol (Adams and Cory, 1998; Green and Reed, 1998; Reed, et al., 1998). Mitochondrial translocation of Bax is one of the critical steps for the induction of apoptosis, however the mechanism is not yet fully understood.

Translocation of Bax from the cytosol to mitochondria is caspase-independent, since caspase-inhibitor pretreatment does not interfere with this process (Goping, et al., 1998). C-terminus hydrophobic residues forming the ninth α-helix of Bax are reported to be involved in the translocation of Bax to the mitochondrial membrane (Suzuki, et al., 2000). In addition, some of BH3-only proapoptotic Bcl-2 family members, such as Bid, are reported to stimulate the membrane insertion of Bax and its oligomerization in mitochondria (Cheng, et al., 2001; Wei, et al., 2001). On the other hand, the N-terminus of Bax functions as a cytosol retention domain, since the deletion of this region allowed Bax to accumulate in the mitochondrial membrane in the absence of apoptotic stimuli (Goping, et al., 1998). These previous observations suggest the presence of the cytosol retention factor(s) and apoptotic stimulation activates Bax protein escape from the factor(s).

### BRIEF SUMMARY OF THE INVENTION

In one embodiment, the present invention is a method of predicting whether cancer cells would respond to therapies which are mediated through Bax-regulated apoptosis, comprising the step of: (a) examining the intensity of the expression of the Bax gene in cancer cells relative to a control, and (b) based on the intensity level, predicting whether the cells will respond to therapies which are mediated through Bax-regulated apoptosis, wherein a high Bax level indicates that one may lower Ku70 levels and increase sensitivity to apoptosis. In a preferred embodiment, one additionally examines the intensity of expression of the Ku70 gene in a cell, preferably by measuring the amount of Ku70-specific mRNA.

In another embodiment, the invention is a method of increasing the sensitivity of cells to therapy, comprising the step of reducing the cells' native Ku70 protein or mRNA level sufficiently so that the cell becomes more sensitive to cancer therapy. Preferably, the reduction is through antisense mRNA methods.

In another embodiment, the invention is a method of treating cell death-related diseases comprising the step of increasing cellular Ku70 protein or mRNA level.

Other objects, features, and advantages are also part of the present invention. One should review the specification, claims, and drawings to fully understand the scope of the present invention.

### BRIEF DESCRIPTION OF THE SEVERAL VIEWS OF THE DRAWINGS

FIG. 1. Ku70 shows cytoprotective activity. FIG. 1A: Scheme of Ku70 full-length, Bax-suppressor clones (clone 1 and 2) obtained by yeast-based functional screening using pGilda-Bax plasmid for Bax expression as reported (Xu, et al., 2000). FIG. 1B: Ku70 suppresses Bax-induced apoptosis in HEK293T cells as well as XIAP. 10⁶ cells were transfected with 1.0 μg of pcDNA3 (Control) or pcDNA3-Bax (Bax) together with 0.5, 1.0, or 2.0 μg of pCMV-2B-Ku70 wt (Ku70 wt) or pcDNA3-Myc-XIAP (XIAP). In “Bax+Vector” group, 1.0 μg pcDNA3-Bax and 2 ug of pCMV-2B were used, respectively (Control and Bax+Vector). All the cells were also co-transfected with 0.5 μg pEGFP for the marking of transfected cells. Apoptosis in the transfected cells was analyzed 24 hours following transfection with Hoechst dye staining of the nucleus as described in Experimental Procedure. FIG. 1C: Time course of the suppression of Bax-induced apoptosis by Ku70 and XIAP in HEK293T cells. 10⁶ cells were transfected with 1.0 μg pcDNA3-Bax and 2.0 μg pCMV-2B (Bax+Vector), pCMV-2B-Ku70 wt (Bax+Ku70 wt), or pcDNA3-Myc-XIAP (Bax+XIAP). In control group, 1.0 μg pcDNA3 and 2.0 μg pCMV-2B were used (Control). Apoptosis in the transfected cells was analyzed at 24 hours (Control) or the indicated time points following transfection. FIG. 1D: The C-terminus of Ku70 suppresses Bax-induced apoptosis in HEK293T cells. 10⁶ cells were transfected with 1.0 μg pcDNA3-Bax (Bax) together with 2.0 μg pCMV-2B (Vector), pcDNA3-Myc-XIAP (XIAP), pCMV-2B-Ku70 wt (Ku70 wt), pCMV-2B-Ku70₁₋₅₃₅ (Ku70₁₋₅₃₅), pCMV-2B-Ku70₄₉₆₋₆₀₉ (Ku70₄₉₆₋₆₀₉), or pCMV-2B- Ku70₅₃₆₋₆₀₉ (Ku70₅₃₆₋₆₀₉). In control group, 1.0 μg pcDNA3 and 2.0 μg pCMV-2B were used (Control). The effects of Ku70 wt on Bak-mediated apoptosis were examined by co-transfection of cells with 1.0 μg pcDNA3-Bak and 2.0 μg pCMV-2B (Vector) or pCMV-2B-Ku70 wt (Ku70 wt). After 24 hours, apoptotic cells were counted as described in FIG. 1B. FIG. 1E: Ku70₁₋₅₃₅ failed to suppress Bax-induced apoptosis. 10⁶ cells were transfected with 1.0 μg pcDNA3-Bax together with 0.5, 1.0, or 2.0 μg pCMV-2B-Ku70₁₋₅₃₅. In control or vector group, 2.0 μg pCMV-2B and 1.0 μg pcDNA3 or 1.0 μg pcDNA3-Bax were used, respectively (Control and Bax+Vector). The number of apoptotic cells was determined as described in FIG. 1B. FIG. 1F: 10⁶ cells were transfected with 1.0 μg pcDNA3-Bax together with 2.0 μg pCMV-2B (Bax+Vector) or pCMV-2B-Ku70₁₋₅₃₅ (Bax+Ku70₁₋₅₃₅). In control group, 1.0 μg pcDNA3 and 2.0 μg pCMV-2B were used (Control). Apoptosis in the transfected cells was analyzed at the indicated periods following transfection as described in FIG. 1C.

FIG. 2A: Ku70 suppressed STS-induced apoptosis in Hela cells. Hela cells (10⁶ cells) were transfected with 0.5 μg pEGFP and 1.0 μg pCMV-2B (Vector), pCMV-2B-Ku70 (Ku70 wt), pCMV-2B-Ku70₁₋₅₃₅ (Ku70₁₋₅₃₅), or pCMV-2B-Ku70₅₃₆₋₆₀₉ (Ku70₅₃₆₋₆₀₉). One day following transfection, cells were treated with 200 nM STS and the number of apoptotic cells was counted as described in FIG. 1 after 24 hours of STS treatment. FIG. 2B: Ku70 suppressed UVC-induced apoptosis. HEK293T cells (10⁶ cells) were transfected with 0.5 μg PEGFP and 1.0 μg pCMV-2B (Vector), pCMV-2B-Ku70 wt (Ku70 wt), or pCMV-2B-Ku70₅₃₆₋₆₀₉ (Ku70₅₃₆₋₆₀₉). One day following transfection, cells were exposed to 200 J/m² of UVC-irradiation. After 24 hours, apoptotic cells were counted as described in FIG. 1. FIG. 2C: Ku70 suppressed Bax-induced Caspase activation. HEK293T cells (10⁶ cells) were transfected with 1.0 μg pcDNA3 and 2.0 μg pCMV-2B (Control), 1.0 μg pcDNA3-Bax and pCMV-2B (Bax+Vector), pCMV-2B-Ku70 wt (Bax+Ku70 wt), or pCMV-2B-Ku70₄₉₆₋₆₀₉ (Bax+Ku70₄₉₆₋₆₀₉). Caspase activity was measured one day following transfection as described in Experimental Procedure. FIG. 2D: Ku70 suppressed STS-induced Caspase activation. Hela cells (10⁶ cells) were transfected with 1.0 μg pCMV-2B (Vector), pCMV-2B-Ku70 wt (Ku70 wt), or pCMV-2B-Ku70₄₉₆₋₆₀₉ (Ku70₄₉₆₋₆₀₉). Caspase activity was assessed as described in Experimental Procedure. FIG. 2E: Ku70 inhibited Cytochrome c release from mitochondria. HEK293T cells (10⁶ cells) were co-transfected with 1.0 μg pcDNA3 and 2.0 μg pCMV-2B (Control), or 1.0 μg pcDNA3-Bax (Bax) and 2.0 μg pCMV-2B (Vector) or pCMV-2B-Ku70 wt (Ku70 wt). Cytochrome c released from mitochondria into cytosol was analyzed by subcellular fractionation followed by Western blot analysis of Cytochrome c (Cyt c) as well as mitochondrial FoF1-ATP-synthase subunit F1α (F1α) as described in Experimental Procedure.

FIG. 3. Lowering Ku70 protein levels sensitized cells to apoptotic stimuli. FIG. 3A-E: The inserted Western-blot results show the confirmation of the down regulation of Ku70 levels by antisense RNA expression. One million of HEK293T cells (A and C) or HeLa cells (B, D, and E) were transfected with 2.0 μg pcDNA3 (Vector) or pcDNA3-reversed cDNA of Ku70 (antisense (AS) Ku70). Twenty-four hrs later, cells were collected and the levels of Ku70 as well as β-Tubulin were examined using total cell lysates (20 ug protein/lane). FIG. 3A: HEK293T cells (10⁶ cells) were transfected with 2.0 μg pcDNA3 (Vector) or pcDNA3-antisense Ku70 (ASKu70). One day following transfection, cells were transfected with 0.5 μg pEGFP and 1.0 μg pcDNA3 (Vector) or pcDNA3-Bax (Bax). One day following the second transfection, the number of apoptotic cells was determined as described in FIG. 1. FIG. 3B: Hela cells (10⁶ cells) were transfected with 0.5 μg PEGFP and 2.0 μg pcDNA3 (Vector) or pcDNA3-antisense Ku70 (ASKu70). One day following transfection, cells were treated with 200 nM STS for 24 hours. The number of apoptotic cells was measured as described in FIG. 1. FIG. 3C: HEK293T cells (10⁶ cells) were transfected with 0.5 μg PEGFP and 2.0 μg pcDNA3 (Vector) or pcDNA3-ASKu70 (ASKu70). One day following transfection, cells were exposed to 200 J/m² of UVC. One day following UVC-irradiation, the number of apoptotic cells was determined as described in FIG. 1. FIGS. 3D and E): Hela cells (10⁶ cells) were transfected with 0.5 μg pEGFP and 2.0 μg pcDNA3 (Vector) or pcDNA3-ASKu70 (ASKu70). One day following transfection, cells were treated with anti-Fas antibody (CH-11) or human recombinant TRAIL at the indicated various concentrations for 24 hours. The number of apoptotic cells was measured as described in FIG. 1. FIG. 3F: Expression levels of Ku70 and β-Tubulin in MEFs were examined by Western blotting. Total cell lysates containing 20 ug protein were analyzed in each lane. FIGS. 3G and H: Examination of sensitivities of Ku70+/− or Ku70−/− MEFs. MEFs derived from Ku70-proficient (Ku70+/+), Ku70-heterozygous (Ku70 +/−) or Ku70-deficient (Ku70−/−) mice were treated with STS (200 nM) or UVC-irradiation (200 J/m²), and apoptotic cells were counted at the indicated periods as described in FIG. 1.

FIG. 4. Interaction of Ku70 and Bax. FIGS. 4A and B: Co-immunoprecipitation of endogenous Ku70 and Bax. HEK293T cells were lysed in the hypotonic buffer without detergent. Immunoprecipitation was also performed in detergent free buffer as described in Experimental Procedure. Immunoprecipitation was performed with (FIG. 4A) anti-Bax rabbit polyclonal antibody or (FIG. 4B) anti-Ku70 mouse monoclonal antibody as described in Experimental Procedure. Pre-immune rabbit serum (NRS) and mouse IgG were used as negative controls. Western blot analyses of pre-immunoprecipitation (Input) and immunoprecipitated samples (IP) were performed by anti-Ku70 monoclonal antibody or anti-Bax polyclonal antibody. (FIG. 4C) Co-immunoprecipitation of GFP-Bax and Ku70. HEK293T cells (10⁶ cells) were transfected with 1.0 μg of PEGFP (GFP), pEGFP-Baxwt (GFP-Baxwt), pEGFP-Bax-ΔN (GFP-BaxΔN), pEGFP-Bax-Δα9 (GFP-BaxΔα9) or pEGFP-Bax-Δα2 (GFP-BaxΔα2) in the presence of 50 μM z-VAD-fmk. One day following transfection, cells were collected and co-immunoprecipitation experiments of GFP-Bax and endogenous Ku70 were performed as described in Experimental Procedure. Anti-GFP polyclonal antibody was used for immunoprecipitation and detection of GFP-fused proteins, and anti-Ku70 monoclonal antibody for the detection of Ku70. (FIG. 4D) Co-immunoprecipitation of Flag-tagged-Ku70 and endogenous Bax. HEK293T cells (10⁶ cells) were co-transfected with 1.0 μg pcDNA3-Bax and 1.0 μg pCMV-2B-control vector (Flag-tagged firefly luciferase), pCMV-2B-Ku70 wt (Flag-Ku70 wt), pCMV-2B-Ku70₁₋₅₃₅ (Flag-Ku70₁₋₅₃₅), pCMV-2B-Ku70₄₉₆₋₆₀₉ (Flag-Ku70₄₉₆₋₆₀₉) or pCMV-2B-Ku70₅₃₆₋₆₀₉ (Flag-Ku70₅₃₆₋₆₀₉) in the presence of 50 μM z-VAD-fmk. Co-immunoprecipitation was performed as described in FIG. 4C, and Western blot of Bax was done with anti-human Bax polyclonal antibody.

FIG. 5A: Ku70 did not suppress Bax-ΔN-induced apoptosis. Bax-deficient Du145 cells (10⁶ cells) were transfected by 1.0 μg of pcDNA3-Bax (Bax) or pcDNA3-Bax-ΔN (BaxΔN), together with 1.0 μg of pCMV-2B (Vector), pcDNA3-Myc-XIAP (XIAP), pcDNA3-Bcl-2 (Bcl-2), pcDNA3-Bcl-XL (Bcl-XL), or pCMV-2B-Ku70 wt (Ku70 wt). All the cells were also co-transfected with 0.5 μg PEGFP for the marking of transfected cells. Cells in the control group received 0.5 μg of pEGFP, 1.0 μg of pcDNA3, and 1.0 μg of pCMV-2B. One day following transfection, apoptosis was detected as described in FIG. 1. FIG. 5B: Ku70 did not suppress STS-induced apoptosis in Bax-deficient cells. Du145 cells (10⁶ cells) were transfected with 1.0 μg pCMV-2B (Vector), pcDNA3-Myc-XIAP (XIAP), pCMV-2B-Ku70 wt (Ku70 wt), or pCMV-2B-Ku70₅₃₆₋₆₀₉ (Ku70₅₃₆₋₆₀₉) together with 0.5 μg pEGFP. One day following transfection, cells were treated with 200 nM STS and the number of apoptotic cells was counted as described in FIG. 1 after 24 hours of STS treatment. In control group, 1.0 μg of pcDNA3 and pCMV-2B were transfected (Control). FIG. 5C: Ku70 wt, but not C-terminus of Ku70 suppressed UVC-induced apoptosis in Bax-deficient cells. Du145 cells (10⁶ cells) were transfected with 0.5 μg PEGFP and 1.0 μg pCMV-2B (Vector), pcDNA3-Myc-XIAP (XIAP), pCMV-2B-Ku70 wt (Ku70 wt), or pCMV-2B-Ku70₅₃₆₋₆₀₉ (Ku70₅₃₆₋₆₀₉). One day following transfection, cells were exposed to 200 J/m² of UVC-irradiation. After 24 hours, apoptotic cells were counted as described in FIG. 1. FIG. 5D: Down regulation of Ku70 did not induce hypersensitivities to STS in Bax-deficient cells. Du145 cells (10⁷ cells) were transfected with 5.0 μg pEGFP and 20 μg pcDNA3 (Vector) or pcDNA3-antisense Ku70 (ASKu70). One day following transfection, 10⁶ cells were collected and the levels of Ku70 as well as β-Tubulin were examined by Western blotting. Remained cells (10⁶ cells for each group) were treated with 200 nM STS for 24 hours or re-transfected with 1.0 ug of pcDNA3 (Vector) or pcDNA3-Bax (Bax). The number of apoptotic cells was counted as described in FIG. 1. FIGS. 5E and F: Ku70 did not suppress apoptosis induced by anti-Fas antibody (Clone CH-11) or human recombinant TRAIL in Hela cells. Hela cells (10⁶ cells) were transfected with 0.5, 1.0, or 2.0 μg pCMV-2B-Ku70 wt (Ku70 wt). One day following transfection, cells were treated with 1 μg/ml anti-Fas antibody or 100 ng/ml TRAIL and the number of apoptotic cells was counted after 24 hours of anti-Fas antibody or TRAIL treatment as described in FIG. 1. In control group, 2.0 μg pCMV-2B were used (Control). The cells in vector group received 2.0 μg pCMV-2B and 1 μg/ml anti-Fas antibody (Fas+Vector) or 100 ng/ml TRAIL (TRAIL+Vector).

FIG. 6. Ku70 sequestered Bax from mitochondria. FIG. 6A: Subcellular localization of Bax and Ku70. HeLa cells (10⁷ cells) were transfected with 10 μg pCMV-2B (Control, STS,) or pCMV-2B-Ku70 (STS+Ku70). One day following transfection, except in the control group (Control), cells were treated by 200 nM STS (STS). One day after STS-treatment, cells were collected and subcellular fractionation was performed as described in Experimental Procedure. The levels of Ku70 (Ku70) and Bax (Bax) in each fraction were analyzed by Western blotting as described in Experimental Procedure. FoF1 ATP synthase subunit α (F1α) and PCNA (PCNA) were used as markers for mitochondrial and nuclear fractions, respectively. HM stands for “Heavy Membrane” fraction containing mitochondria. FIG. 6B: Ku70 overexpression increased the capacity of Bax in the cytosol. HEK293T cells (10⁷ cells) were transfected with 5.0 μg pcDNA3 and 10 μg pCMV-2B (Control), 5.0 μg pcDNA3-Bax and 10 μg pCMV-2B (Bax+Vector), or 5.0 μg pcDNA3-Bax and 10 μg pCMV-2B-Ku70 (Bax+Ku70). One day following transfection, cells were collected and subcellular fractionation and Western blot analyses of Bax and mitochondrial FoF1-ATP-synthase subunit F1α (F1α) were performed as described in Experimental Procedure. FIG. 6C: Caspase-independent disappearance of Ku70 during apoptosis. Hela cells (10⁶ cells) were treated with 200 nM STS in the absence (STS) or presence of z-VAD-fmk (STS+z-VAD). One day following the treatment, cells were collected and fractionated as described in Experimental Procedure. Cytosol fractions (20 μg protein) were separated by SDS-PAGE and analyzed by Western blotting for Ku70 (anti-Ku70 monoclonal antibody, BD-Pharmingen) and β-Tubulin (anti-β-Tubulin monoclonal antibody, BD-Pharmingen) levels. The effect of z-VAD-fmk was confirmed by its suppression of apoptosis. The percentages of apoptotic cells were 3±1% in control, 49±4% in STS-treated cells, and 11±3% in STS- and z-VAD-fmk-treated cells. FIG. 6D: Lowering Ku70 levels increased mitochondrial Bax levels, but reduced nuclear Bax levels. Antisense Ku70 RNA was expressed in HEK293T cells as described in FIG. 2. Subcellular fractionation and Western blot analyses were performed as described in FIG. 6A. FoF1 ATP synthase subunit α (F1α) and PCNA (PCNA) were used as internal controls for mitochoridrial and nuclear fractions, respectively. FIG. 6E: Subcellular localization of Bax in Ku70-deficient MEFs. MEFs derived from wild-type (Ku70+/+) or Ku70-knockout mice (Ku70−/−) were analyzed as described in FIG. 6A. Anti-mouse Bax antibody was used for Bax detection of MEFs. F1α and PCNA were used as internal controls for mitochondrial and nuclear fractions, respectively. FIG. 6F: Time course of mitochondrial translocation of Bax in MEFs during apoptosis. MEFs derived from wild-type (Ku70+/+) or Ku70-deficient (Ku70−/−) mice were treated with STS (200 nM) and cells were analyzed at indicated various periods after treatment as described in FIG. 6A. FoF1-ATP-synthase subunit α (F1α) was used as a maker of mitochondria-containing heavy membrane (HM) fraction.

FIG. 7A: Subcellular localization of Bax and Ku70. HEK293T cells (10⁷ cells) were transfected with 10 ug pCMV-2B (Control, UV, and UV+z-VAD) or pCMV-2B-Ku70 (UV+Ku70). One day following transfection, except in the control group (Control), cells were exposed to UVC-irradiation in the absence (UV or UV+Ku70) or the presence of 50 uM z-VAD-fmk (UV+z-VAD). One day following UVC-irradiation (200 J/m²), cells were collected in lysis buffer (200 ul) and subcellular fractionation was performed as described in Experimental Procedure. HM stands for “Heavy Membrane” fraction enriched with mitochondria. The effect of z-VAD-fmk was confirmed by its suppression of apoptosis. The percentages of apoptotic cells were 2±2% in control, 42±5% in UV-treated cells, and 9±1% in UV- and z-VAD-fmk-treated cells. FIG. 7B: Ku70 suppresses the relocalization of Bax during apoptosis. HEK293T cells were transfected with pCMV-2B-vector (Control and UV+z-VAD) or pCMV-2B-Ku70 (UV+Ku70). Except “control” cells were treated by UVC-irradiation (200 J/m²). One day after UVC-irradiation, cells were fixed and the double staining of Ku70 and Bax were performed as described in Experimental Procedure. Control Group: Ku70 is detected both in the cytosol and the nucleus. Large proportion of Bax distributes in the cytosol and Bax is also detected in the nucleus. UV+z-VAD Group: Ku70 in the cytosol disappeared. Bax staining pattern changes from cytosolic distribution to punctuated mitochondria-like one. UV+Ku70 Group: Ku70 overexpression increased Ku70-signals both in the cytosol and the nucleus. Ku70 suppressed Bax translocation and Bax remains in the cytosol. Ku70 (536-609), which does not have DNA-repair function, also inhibited Bax relocalization induced by UVC-irradiation (not shown).

FIG. 8. Expression levels of Ku70 and Bax in fourteen cancer cell lines, and the reduction of Ku70 levels by antisense Ku70 RNA expression. FIG. 8A and B: Expression levels of Ku70 and Bax in cancer cells were analyzed by Western blotting. Cell lysates (20 ug protein) were applied to each lane. HeLa cells were used as the “standard” cell line. The cancer cell lines used are glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP and Du145), and breast cancer cells (MCF-7 and MDA-MB-468). FIG. 8C: Antisense Ku70 reduced Ku70 levels without the significant change in the levels of cell death regulators. U87-MG (glioma cell line) and HT-1080 (fibrosarcoma) were transfected with the plasmid encoding antisense Ku70 RNA (reversed Ku70 cDNA is subcloned in the vector to express antisense Ku70 RNA) (“AS Ku70”) or the vector plasmid (pcDNA3 vector) (“Control”). One day following the transfection, cells were collected and the levels of Ku70 and cell death regulator Bcl-2 family proteins (Bax, Bcl-2, and Bcl-XL) were examined by Western blotting.

FIG. 9. Reduction of Ku70 levels by antisense Ku70 RNA enhances the mitochondrial translocation of Bax in cancer cells. FIGS. 9A and B: Cancer cells were transfected with the plasmid encoding antisense Ku70 RNA (pcDNA3-antisense Ku70) (“AS Ku70”) or the vector plasmid (pcDNA3) (“Control”). One day following the transfection, cells were treated by 20 uM etoposide for 24 hours and cells (10⁷ cells) were collected in the lysis buffer (200 ul). Subcellular fractionation was performed to collect the fractions of the cytosol (“Cytosol”) and the heavy membrane (“HM”). The heavy membrane faction contains mitochondria. The 20 ug protein samples of the total cell lysates and the cytosol fraction were analyzed by Western blotting for the levels of Ku70 and Bax, respectively. The proportion of 20 ug protein samples in the total cytosol fraction was calculated and the same proportion of the samples from the total heavy membrane fractions were used for Western analysis of Bax levels. Please see detail in “the method section”. The cell lines examined were: glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP), and breast cancer cells (MCF-7 and MDA-MB-468).

FIG. 10. Antisense Ku70 RNA enhances etoposide-induced apoptosis in cancer cell. FIG. 10A-L: Cancer cells (the name of cell lines is indicated in each graph) were transfected with the plasmid encoding antisense Ku70 RNA (pcDNA3-antisense Ku70) (“AS Ku70”) or the vector plasmid (pcDNA3) (“Control”). All cells were also co-transfected with the plasmid encoding Green Fluorescent Protein (GFP) (PEGFP plasmid) for the detection of the transfected cells by GFP expression. One day following the transfection, cells were treated by 20 uM etoposide for 48 hours. The percentages of apoptotic cells were counted in GFP-expressing cells by staining the nucleus with Hochst-dye on day 1 and 2 of the culture. The cell lines examined were: glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP), and breast cancer cells (MCF-7 and MDA-MB-468).

FIG. 11. Antisense Ku70 RNA enhances cisplatin-induced apoptosis in cancer cell. FIG. 11A-L: Cancer cells (the name of cell lines is indicated in each graph) were transfected with the plasmid encoding antisense Ku70 RNA (pcDNA3-antisense Ku70) (“AS Ku70”) or the vector plasmid (pcDNA3) (“Control”). All cells were also co-transfected with the plasmid encoding Green Fluorescent Protein (GFP) (PEGFP plasmid) for the detection of the transfected cells by GFP expression. One day following the transfection, cells were treated by 20 uM cisplatin for 48 hours. The percentages of apoptotic cells were counted in GFP-expressing cells by staining the nucleus with Hochst-dye on day 1 and 2 of the culture. The cell lines examined were: glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP), and breast cancer cells (MCF-7 and MDA-MB-468).

FIG. 12. Antisense Ku70 RNA enhances doxorubicin-induced apoptosis in cancer cell. FIG. 12A-L: Cancer cells (the name of cell lines is indicated in each graph) were transfected with the plasmid encoding antisense Ku70 RNA (pcDNA3-antisense Ku70) (“AS Ku70”) or the vector plasmid (pcDNA3) (“Control”). All cells were also co-transfected with the plasmid encoding Green Fluorescent Protein (GFP) (pEGFP plasmid) for the detection of the transfected cells by GFP expression. One day following the transfection, cells were treated by 1 uM doxorubicin for 48 hours. The percentages of apoptotic cells were counted in GFP-expressing cells by staining the nucleus with Hochst-dye on day 1 and 2 of the culture. The cell lines examined were: glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP), and breast cancer cells (MCF-7 and MDA-MB-468).

FIG. 13. Antisense Ku70 RNA enhances the suppression of cancer cell growth by anti-cancer drug. FIG. 13A-D: Two glioma cell lines (A and B: U87-MG, C and D: T98-G) were transfected with the plasmid encoding antisense Ku70 RNA (pcDNA3-antisense Ku70) (“Etposide+AS Ku70”) or the vector plasmid (pcDNA3) (“Etoposide+Vector”). One day following the transfection, etoposide (20 uM) was added to the culture and the cells were culture for three week. In the “Control” group, cells were transfected with the vector plasmid and cultured without etoposide. After the incubation, cells were stained by heamatoxyline to visualize the colonies on the culture dish (A and C). The number of the colonies in the same area in each plates was counted and the percentages of them (the number in the control group is designated as 100%) were shown in B and D. FIG. 13E: A glioma cell line (T98G) were transfected with the plasmids encoding antisense Ku70 (pcDNA3-antisense Ku70) and Green Fluorescent Protein (pEGFP). One day after the following plasmid transfection, cells were treated by etoposide (20 uM) for 24 hours and the nuclei of the cells were stained by Hochst-dye (FIG. 13E left panel). The transfected cells (green cells in the right panel) show typical apoptotic nuclei (nuclear fragmentation) (right panel).

FIG. 14. Reduction of Ku70 levels does not increase cell killing activities of anti-cancer drugs in Bax-deficient cells (Du145). FIG. 14A: The expression levels of Ku70 and Bax in Du145 cells. Bax-deficient prostate cancer cell line, Du145, were transfected with the plasmid encoding antisense Ku70 RNA (“AS Ku70”) or the vector plasmid (“Control”). One day following the transfection, cells were treated by etoposide (20 uM). One day after the addition of etoposide into the culture, cells were collected and subcellular fractionation was performed as described in FIG. 9. Total cell lysates (20 ug protein) were subjected to Western blotting of Ku70 (upper panel). Bax leveles in the samples of the fractions of the cytosol (“Cytosol”) and the heavy membrane (“HM”) were analyzed by Western blotting (middle and lower panels). FIG. 14B-E: Du145 cells were transfected with the plasmid encoding antisense Ku70 RNA (“AS Ku70”) or the vector plasmid (“Control”). All cells were also co-transfected with the plasmid encoding Green Fluorescent Protein (GFP) (pEGFP plasmid) for the detection of the transfected cells by GFP expression. One day following the transfection, etoposide (20 uM), cisplatin (20 uM), doxorubicin (1 uM), or TRAIL (100 ng/ml) was added to the culture, and cells were culture for 48 hours. The percentages of apoptotic cells were counted in GFP-expressing cells by staining the nucleus with Hochst-dye on day 1 and 2 of the culture. FIG. 14F: TRAIL does not induce the mitochondrial translocation of Bax. T98-G (glioma cell line) and Hep3B (hepatoma cell line) cells were cultured for 24 hours in the absence (“Control”) or the presence (“TRAIL”) of 100 ng/ml TRAIL. After the culture, cells were collected and subcellular fractionation was performed as described in FIG. 2. The samples of the cytosol fraction (20 ug protein) (“Cytosol”) and the equivalent proportion of heavy membrane fraction (“HM”) were subjected to Western analysis of Bax.

FIG. 15. Antisense Ku70 RNA does not affect TRAIL-induced apoptosis in cancer cells. FIG. 15A-L: Cancer cells (the name of cell lines is indicated in each graph) were transfected with the plasmid encoding antisense Ku70 RNA (pcDNA3-antisense Ku70) (“AS Ku70”) or the vector plasmid (pcDNA3)(“Control”). All cells were also co-transfected with the plasmid encoding Green Fluorescent Protein (GFP) (PEGFP plasmid) for the detection of the transfected cells by GFP expression. One day following the transfection, cells were treated by 100 ng/ml TRAIL for 48 hours. The percentages of apoptotic cells were counted in GFP-expressing cells by staining the nucleus with Hochst-dye on day 1 and 2 of the culture. The cell lines examined were: glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP), and breast cancer cells (MCF-7 and MDA-MB-468).

### DETAILED DESCRIPTION OF THE INVENTION

We performed yeast-based functional screening of cell death suppressor genes and cloned the Ku70 gene from both human and mouse cDNA libraries. The sequence of the Ku70 gene may be found in GenBank at accession no. NM_{—}001469 and is also found in Chan, et al., 1989 and Reeves and Sthoeger, 1989. Chan, et al., 1989 and Reeves and Sthoeger, 1989 are incorporated by reference herein.

Our screening system was developed based on cell death-inducing activity of human Bax protein in budding yeast. Bax is a cyto-destructive member of Bcl-2 family proteins known to be a key protein group to regulate cell suicide called programmed cell death or apoptosis. The DNA sequence of the Bax gene is found in GenBank at accession no. L22473 and in Oltvai, et al., 1993. Oltvai, et al. is incorporated by reference herein.

Our observations described below suggest the presence of new physiological function of Ku70, namely anti-cell death function by suppressing the activity of Bax. Our new findings provide new strategies to use Ku70-related biochemical products to treat cell death-related diseases, such as cancer and ischemia-induced cell death in nervous and cardiovascular systems, and as a diagnostic tool.

One important character of Ku70-related products (genes, oligonucleotides, and peptides) is low risk of side effects. Increase of Ku70 level itself has no toxic activity to the cells, and it protects cells from death, therefore this type of treatment will not have immediate damage to the tissue. Lowering Ku70 levels can be expected to sensitize the cells to naturally occurring DNA-damage, however other DNA-repair proteins seem to compensate the loss of Ku70, since complete deletion of Ku70 gene in mice doe not cause lethal effects. In fact, antisense mRNA treatment did not induce apoptosis itself, but only sensitize the cells to cell death treatment such as anti-cancer drugs. This character of Ku70-related treatment may serve new way of chemotherapy and radiation therapy to the patient.

In addition, Ku70 is evolutionary conserved protein from yeast to human, and is expressed ubiquitously in the human body. Therefore, Ku70-related treatment to regulate cell death may be applied to the many types of health problems in many tissues.

Increase of Ku70 Level

In one embodiment of the present invention, the newly discovered anti-Bax activity of Ku70 can be used for the treatment of cell death-related diseases. As described above, increase of cellular Ku70 protein level by gene transfer methods encoding Ku70 confers resistance to cytotoxic stimuli to the cells. These strategies may be directly applied, for example, to rescue the cells susceptible to death during the reperfusion treatment after ischemia in the brain and heart. Since HIV-induced lymphocytes death has been reported to involve Bax (Ferri, et al., 2000), similar method may be utilized to rescue HIV-infected lymphocytes. We envision that increase of cellular Ku70 protein level will confer resistance to cytotoxic stimuli to cells at both the cellular and organ/tissue level. Therefore, one may choose to treat a population of cells or may choose to treat a patient.

Ku70 protein levels in cells or tissues can be increased by the commonly used methods in gene therapy, such as by directly injecting an expression plasmid encoding the Ku70 protein or infecting with virus vectors (both DNA and RNA virus types) encoding Ku70 to the target cells, tissue, or organs. One would wish to modulate the Ku protein level to a sufficient amount such that resistance to cytotoxic stimuli may be measured, as demonstrated below in the Examples.

Although the mechanism of Ku70 proteolysis has not been elucidated, treatment of cells or tissues by the inhibitors of Ku70-proteases may be a preferred method in the future. In addition, one might also use methods to increase the levels of the transcription factors that initiate Ku70 gene expression. The increase of these factors may be achieved by gene therapy methods using expression plasmids or the virus vectors encoding their genes.

We envision that the method described above would be particularly useful in treating cells and cell populations, such as stem cells, platelets or white blood cells, that are to be stored for an indeterminate period of time and, thus, at risk for cell death. In this embodiment, the invention is a method of treating solid organs or cells, such as blood cells, platelets, or ischemic cells or tissues, either in vitro or in vivo, to increase Ku70 levels, thru Ku70 mRNA delivery alone or with a vector, Ku70 protein delivery, or up-regulation of the Ku70 gene, to prolong survival of the cells or organ during periods of stress such as hypoxia or apoptosis.

One of skill in the art may obtain a Ku70-encoding sequence in numerous ways using the references for the Ku70 sequence described above. Most typically, Ku70 cDNA can be obtained by RT-PCR using mRNA from human cells such as HeLa cells. Ku70 is ubiquitously expressed in human cells, so most human cells can be the source of Ku70 mRNA. Appropriate primers may be designed from the sequences described above.

By “cell death-related diseases,” we mean degenerative diseases including development failure (abnormal shape or the function of the organs due to the genetic mutation, virus infection, or toxins); ischemia induced tissue damage in the brain (stroke), the heart (heart attack), the kidney, and other organs; re-perfusion induced tissue damage after stroke, heart attack, or renal blood flow failure; cold and heat stress-induced tissue damage; UV-exposure-induced tissue damage; infection-induced tissue damage by virus, bacteria, or other parasitic organisms; toxin-induced tissue damage; and aging.

Decrease in Ku70 Level

In another embodiment of the present invention, decrease of a cells' native Ku70 level, for example by antisense mRNA methods, sensitizes the cell to the death stimuli. This method can be utilized to improve the efficiency of anti-cancer treatment, such as the chemotherapy with SULINDAC and CISPLATIN or X-ray-irradiation, as these treatments are known to activate Bax-mediated cell death pathway. These observations suggest that methods to decrease Ku70 levels in cancer cells can be used (1) to enhance the effectiveness of chemotherapy and radiation therapy to eliminate malignant cancer cells and (2) to lower the doses of anti-cancer drugs for patients reducing the risk of side-effects of these drugs.

Our Examples below demonstrate that antisense Ku70 RNA was effective in increasing the sensitivity of cells to anti-cancer drugs in glioma cells, colon cancer cells, prostatic cancer cells, fibrosarcoma, and cervical cancer cells. These results clearly indicate that the method(s) described herein and other methods, both those used in our laboratory and those used by other workers, of decreasing Ku70 protein level will be applicable to numerous types of cancer cells.

One of skill in the art would understand that there are a variety of molecular biological methods to decrease a particular protein level in either a patient or an individual's cells. Most typically, one would decrease Ku70 levels by transfecting or injecting a plasmid or a virus (RNA or DNA viruses) that expresses antisense Ku70 RNA (effective antisense RNA, such as reversed full-length Ku70 RNA, or short interference RNA (siRNA)). Injecting oligonuceotide, DNA-zyme or RNA-zyme that inhibit Ku70 gene transcription. Silencing factor of Ku70 transcription has not been identified neither, however, the gene therapies increasing the silencing factor may be also possible. Other methods may include the use of antisense oligonucleotides, DNA-zymes, RNA-zymes, and RNAi, that inhibits transcription of Ku70 protein from mRNA. The Ku70 proteases and its enhancer can be also useful to decrease Ku70 protein level in cancer cells.

One would identify a human cancer patient and use molecular biological techniques known to one of skill in the art to decrease the cancer cell populations native Ku70 levels. For example, in a patient with colon cancer, one would attempt to treat the colon cancer cells with antisense Ku70 RNA so that a decrease in the cells' native Ku70 protein level can be measured. We envision that any decrease in the cells' native Ku70 level will enhance treatment with chemotherapy agents described above.

Examination of Ku70 and Bax Levels

We have also found that the examination of Ku70 and Bax levels in cancer cells, preferably the combined examination, can predict the effectiveness of commonly used anti-cancer treatments to induce cell death in cancer cells. This method would be useful to predict the effectiveness of cancer therapy or to design a strategy of cancer therapy.

We found that when the levels of Ku70 protein or RNA are high in cancer cells with normal levels of Bax, these cells are resistant to anti-cancer treatments stimulating Bax-mediated cell suicide signals, treatments such as CISPLATIN, ETOPOSIDE, and UV/X-ray treatments. Lowering Ku70 by antisense Ku70 RNA in the cancer cells expressing high levels of Ku70 and Bax sensitizes these cells to anti-cancer treatments. However, in cancer cells with low levels of Ku70 and/or Bax, such as U373-MG (glioma), A172 (glioma), and HCT116 (colon cancer), lowering Ku70 levels is less effective to increase the sensitivity to anti-cancer drugs. Therefore, the combined examination of the expression levels of Ku70 and Bax mRNA or protein levels is a useful method to predict the effectiveness of commonly used anti-cancer treatments that stimulate Bax-mediated signals and anti-cancer therapy methods (i.e. lowering Ku70 levels in cancer cells).

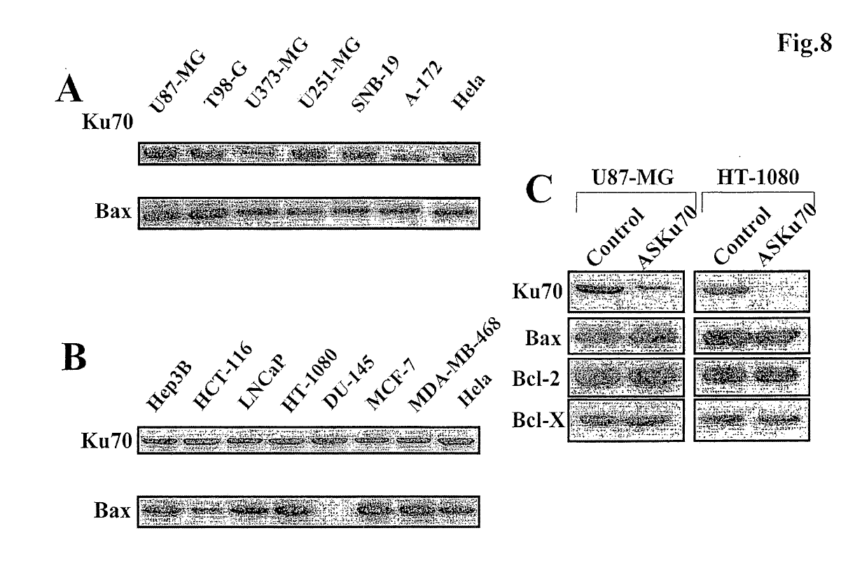

Therefore, in one embodiment, the present invention comprises examining the intensity of the expression level of the Bax and/or Ku70 genes (at either the RNA or protein level) in a cell and predicting whether cells might respond to therapies which are mediated through Bax-regulated apoptosis. “High” and “low” protein levels typically correspond to band intensity in a Western blot type gel system and are relative to commonly used cell lines, such as Hela cells. In a preferred version of the invention, one would compare a test tumor sample to the same cell type to determine whether the Bax and/or Ku70 levels are “high” or “low”. For example, if one is examining a glioma cell tumor, one would preferably compare Bax and/or Ku70 RNA or protein levels in the glioma cell lines listed in FIG. 8.

A preferred embodiment of the comparison method is as follows: Typical methods to examine the levels of Ku70 and Bax protein and mRNA include measuring mRNA levels by DNA-chip, RT-PCR, Northern-Blot analysis, and variations of these technologies, and measuring protein levels by Western blot, dot blot, FACS, immunohistochemistry, and variations of these methods.

If the Bax level is high in cells, one can predict that lowering Ku70 levels may result in increased sensitivities to apoptosis. By examining the Bax level and/or the Ku70 level in a specific tumor, one can determine whether the expression of either can be lowered. Lowering the expression of Ku70 via chemotherapy and/or an antisense RNA molecule results in the hypersensitivities to cancer therapy stimulating Bax-mediated apoptosis.

If the cancerous cell type is one which already has a low expression level of Bax and Ku70, then we predict that drugs which work through Bax-mediated apoptosis, such as CISPLATIN and ETOPOSIDE, would not be effective against that tumor and be contraindicated. However, if Bax and Ku70 are high in a particular tumor, then a chemotherapy which works by decreasing the expression of Ku70 levels will be an appropriate choice.

Some examples of predictions: If Bax levels are low and Ku70 levels are low, then treating with drugs that lower Ku70 will not change cells' sensitivity to treatment. If Bax levels are high (or at least at normal level) and Ku70 levels are high (or at least at normal level), then treating with drugs that lower Ku70 will enhance the effectiveness of Bax-mediated cancer killing. If Bax levels are high and Ku70 levels are low, then treating with drugs which lower Ku70 level may not work to increase the killing of cancer cells.

### EXAMPLES

I. Ku70 Prevents Mitochondrial Translocation of Bax.

We report here that Ku70, a subunit (70 kDa) of Ku-complex comprising Ku70 and Ku80 (80 kDa subunit), has a function to prevent mitochondrial translocation of Bax in normal cells. Ku70 localizes both in the cytosol and the nucleus. Ku70/Ku80-complex has been known to play important roles in DNA-repair in the nucleus (Khanna and Jackson, 2001; Walker, et al., 2001). We found that cytosolic Ku70 binds Bax and inhibits the mitochondrial translocation of Bax. The C-terminus of Ku70, which cannot form a complex with Ku80, interacts with Bax and is sufficient to rescue cells from Bax-mediated apoptosis. In addition, the N-terminus of Bax is required for the interaction with Ku70, which is consistent with the previous finding that the N-terminus of Bax is the cytosol retention domain (Goping, et al., 1998). The present data suggests that Ku70 plays a cytoprotective role as an inhibitor of Bax in the cytosol in addition to its previously known roles in DNA repair.

Ku70 was Identified as a New Bax-Suppressor in Yeast-Based Functional Screening

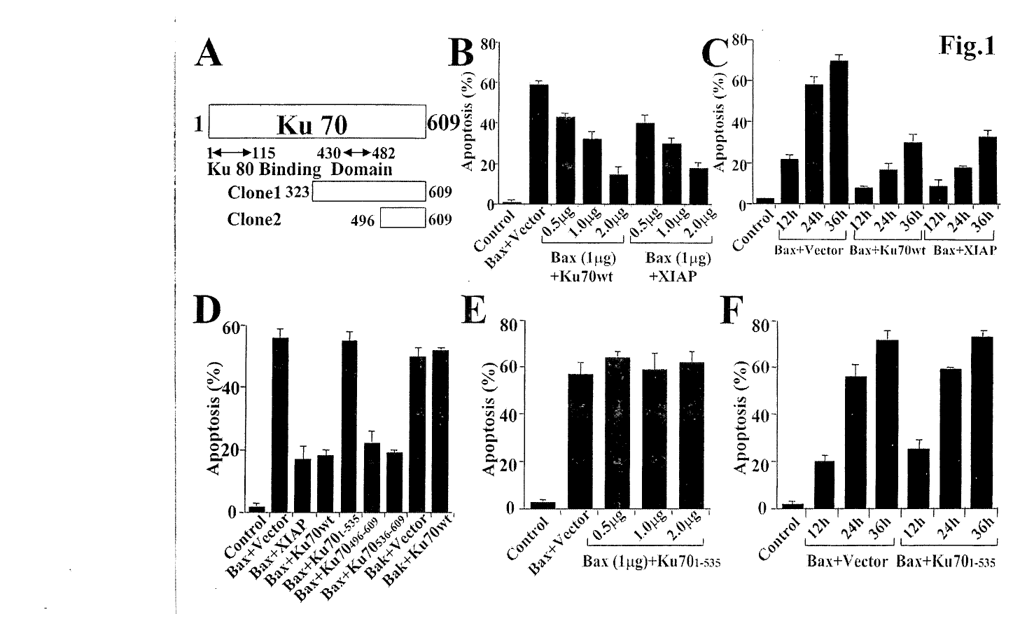

We performed a search for Bax inhibitors using a yeast-based functional screening system (Xu, et al., 2000; Xu and Reed, 1998), and cloned human Ku70 as a potential Bax suppressor protein. Ku70 is the 70 kDa subunit of Ku antigen, a heterodimeric complex composed of Ku70 as well as Ku80 protein (Walker, et al., 2001). Ku70 has been localized to both the cytosol and nucleus (Fewell and Kuff, 1996). Ku is expressed ubiquitously in mammalian cells, and plays an essential role in nonhomologous DNA double-strand break (DSB) repair (Walker, et al., 2001) (Khanna and Jackson, 2001). The heterodimerization domains between Ku80 and Ku70 are localized to amino acids 1-115 and 430-482 in Ku70 (Wang, et al., 1998) (FIG. 1A).

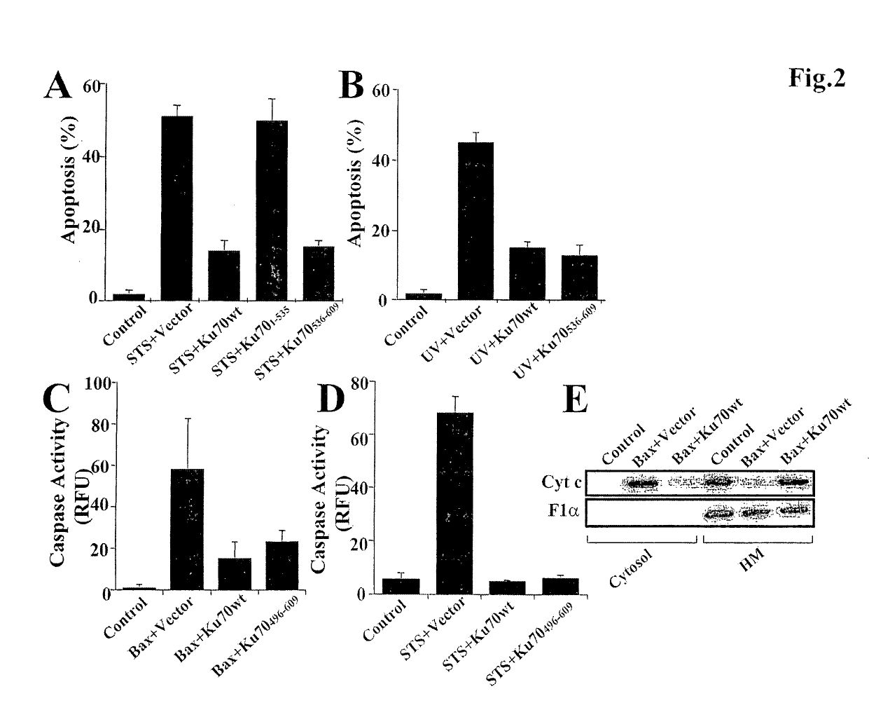

We constructed yeast expression cDNA libraries using mRNA from HeLa cells and mouse brain tissue. Yeast-based functional screening of Bax inhibitors was performed as previously reported (Xu, et al., 2000; Xu and Reed, 1998), and two individual clones were identified as Bax suppressors encoding amino acids 323-609 (clone 1; HeLa cell library) and 496-609 (clone 2; mouse brain library) of Ku70 (FIG. 1A). Human Ku70 mutant constructs, together with full-length human Ku70, were made corresponding to the mouse sequence of clone 2, and tested for its ability to inhibit Bax activity in mammalian cells (FIG. 1). Full-length Ku70 suppressed Bax-induced apoptosis (FIG. 1B and C) and Caspase activation (FIG. 2C) in HEK293T cells as efficiently as XIAP, a potent cytoprotective protein that inhibits Caspase activity (Deveraux, et al., 1997). Ku70 also attenuated Staurosporine (STS) induced apoptosis (FIG. 2A) and Caspase activation (FIG. 2D) in HeLa cells, and in UVC-irradiation-induced cell death in HEK293T cells (FIG. 2B). Interestingly, the Ku70 mutant construct encoding amino acids 496-609 of Ku70 (Ku70₄₉₆₋₆₀₉), which was equivalent to “Bax-inhibitor clone 2” and lacked Ku80-binding domain, retained the cytoprotective activities against Bax-, STS-, and UVC-induced cell death (FIGS. 1D, 2C, and 2D). In addition, deletion of the C-terminal 74 amino acids of Ku70 (Ku70₁₋₅₃₅) resulted in loss of Ku70's cytoprotective activity against Bax-expression and STS, whereas this C-terminal region of Ku70 (Ku70₅₃₆₋₆₀₉) was sufficient to block Bax-, STS-, UVC-induced apoptosis (FIGS. 1D-F, 2A, and 2B). Taken together, the C-terminal region of Ku70 appears to be required for Ku70's cytoprotective function. Among pro-apoptotic Bcl-2 family members, Bax and Bak play a key role in apoptosis induction, as evident by the fact that double deletion of these genes in mice resulted in the resistance of derived cells to several cell death stimuli known to trigger mitochondria-dependent apoptosis (Wei, et al., 2001). In this regard, Ku70 could not suppress Bak-induced cell death in HEK293T cells, suggesting that Ku70 specifically controls Bax-mediated apoptotic signaling (FIG. 1D).

Cytochrome c release from mitochondria induced by Bax-expression was attenuated by Ku70 expression (FIG. 2E). Ku70 also blocked Cytochrome c release from mitochondria in STS-treated HeLa cells and in UVC-irradiated HEK293T cells (data not shown). These results indicate that Ku70 suppresses cell death at an early step in apoptosis, the signals upstream of mitochondrial Cytochrome c release.

Endogenous Ku70 Plays Cytoprotective Roles

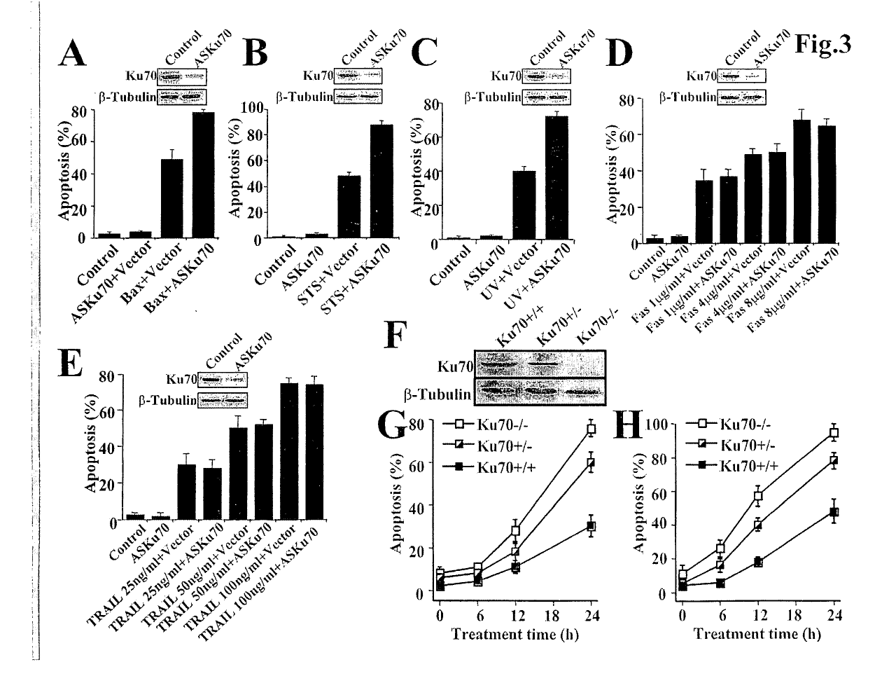

To confirm the cytoprotective role of endogenous Ku70, we examined the effects of antisense-Ku70 RNA expression in HEK293T and HeLa cells. Antisense Ku70 cDNA was subcloned into the pcDNA3 mammalian expression vector and it significantly reduced the Ku70 protein level in HEK293T and HeLa cells as shown in FIG. 3A-E. The expression of antisense Ku70 RNA in these cells resulted in hypersensitivity to Bax-mediated apoptosis induced by Bax-expression, STS or UVC-irradiation (FIG. 3A-C). Furthermore, mycoplasma-free, SV40-transformed mouse embryonic fibroblasts (MEFs) derived from Ku70-deficient mouse also showed increased sensitivities to apoptotic stimuli, such as STS and UVC-irradiation, in contrast to genetically matched Ku70-proficient MEFs (FIG. 3F-H). On the other hand, antisense Ku70 RNA treatment did not change the sensitivities of HeLa cells to “Death Receptor-mediated apoptosis”, such as Fas- and TRAIL-induced apoptosis (FIGS. 3D and E), suggesting that the hypersensitivities to Bax-mediated apoptosis induced by Ku70-deficiency (FIG. 3A-C) were not due to the non-specific cellular damage. These results suggest that Ku70 has a physiological role as an inhibitor of apoptosis.

Ku70 Interacts with Bax

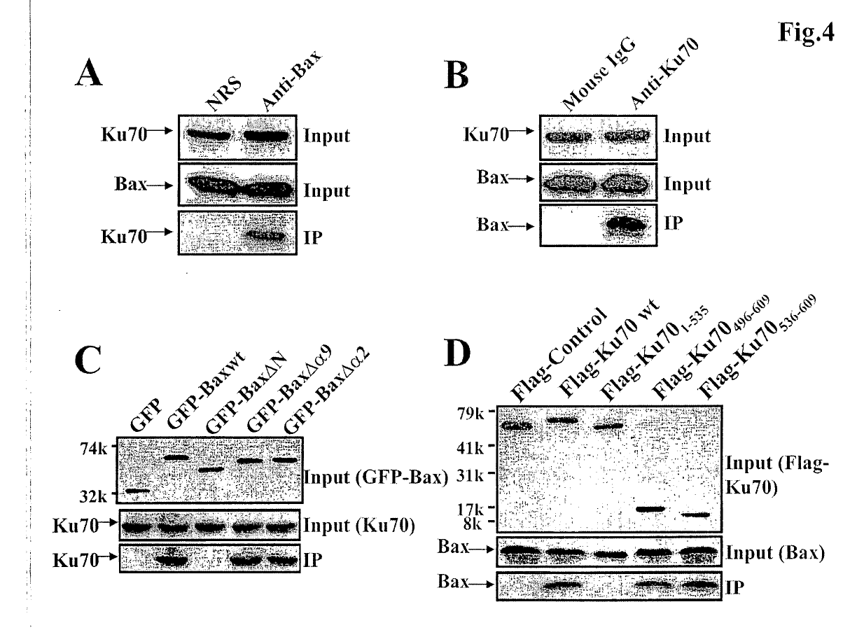

We found that endogenous Ku70 and Bax co-immunoprecipitate each other (FIGS. 4A and B, Supplemental data-A-D), suggesting that Ku70 interacts with Bax. Co-immunoprecipitation experiments of endogenous Ku70 and Bax were performed in the Chaps-based buffer for total cell lysates (Supplemental data C and D) and detergent free buffer for cytosol fraction (FIGS. 4A and B, Supplemental data A and B), according to the previous report showing the effects of the detergents used in the buffer for immunoprecipitation (Hsu and Youle, 1998). Previous studies showed that the presence of certain types of detergent, except Chaps, causes artifact interaction of the proteins among Bcl-2 family proteins in co-immunoprecipitation experiments (Hsu and Youle, 1998). Therefore, the interaction experiments in this study were performed either in Chaps-based buffer or detergent free buffer.

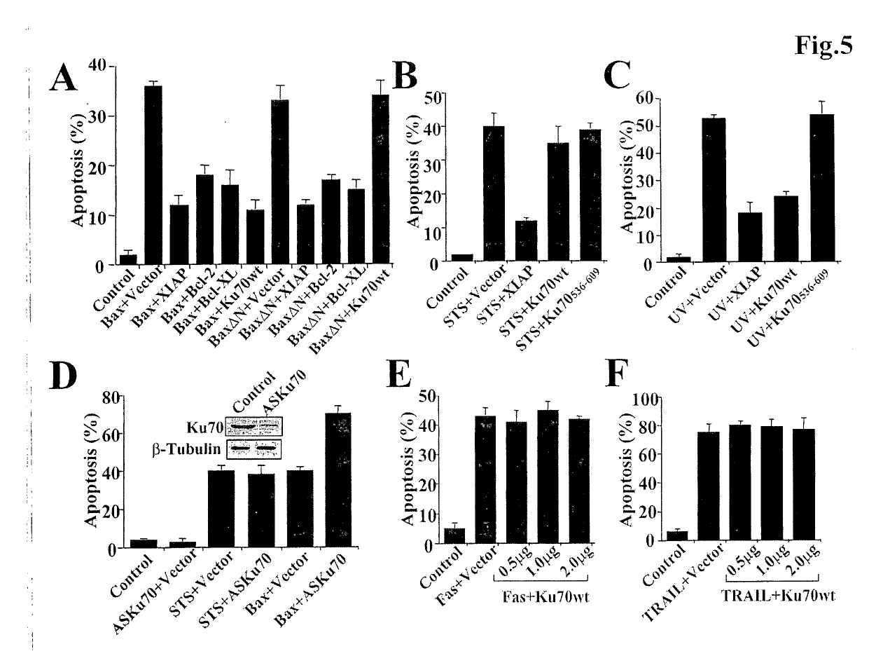

For the identification of the binding domain of Bax with Ku70, we examined the binding activities of several deletion mutants of Bax fused with GFP in total cell lysates (FIG. 4C). Endogenous Ku70 was pulled down by GFP-tagged-Bax (full-length) as well as by deletion mutants of the second α-helix (containing “death-inducing” BH3 domain) (Gross, et a., 1999; Reed, 1997b), and the ninth α-helix (transmembrane domain, mitochondria targeting domain) (Suzuki, et al., 2000) but not by the mutants lacking N-terminus sequences (53 amino acids) prior to the second α-helix (BaxΔN). These results suggest that the N-terminus of Bax is required for its interaction with Ku70. We also examined whether the C-terminus of Ku70, which retains cytoprotective activity, has the activity to bind Bax. Flag-tagged Ku70, Ku70₄₉₆₋₆₀₉, and Ku70₅₃₆₋₆₀₉, but not Ku70₁₋₅₃₅, bound to Bax, suggesting that the Bax-binding domain localized in the C-terminus of Ku70 (FIG. 4D). Furthermore, Ku70 did not suppress apoptosis-inducing activity of BaxΔN in the cells lacking endogenous Bax (Du145, Bax-deficient prostate cancer cell line) (FIG. 5A) (Rampino, et al., 1997). These results suggest that the N-terminus of Bax and the C-terminus of Ku70 are required for the interaction of Bax and Ku70.

C-Terminus of Ku70 does not Suppress Apoptosis in Bax-Deficient Cells

To examine whether Ku70 inhibits cell death signals other than Bax-mediated signals, we examined the anti-apoptotic activity of Ku70 in Bax-deficient cells (a prostate cancer cell line, Du145) (Rampino, et al., 1997) (FIGS. 5B and C). STS is known to trigger mitochondria-dependent cell death pathway through proapoptotic Bcl-2 family proteins such as Bax and Bak, since the double knockout of Bax and Bak conferred the cells resistant to STS (Wei, et al., 2001). Thus, STS is expected to induce apoptosis through pro-apoptotic Bcl-2 family proteins other than Bax, such as Bak, in Bax-deficient cells (Rampino, et al., 1997; Wei, et al., 2001). Ku70, which protected Bax-expressing cells (HEK293T and HeLa cells) from STS (FIGS. 1 and 2), did not suppress STS-induced apoptosis in this Bax-deficient cell line (FIG. 5B). Since full-length Ku70 has an activity to enhance UV-damaged DNA-repair, full length Ku70 (Ku70 wt) overexpression can attenuate UVC-irradiation-induced cell death regardless of Bax expression (FIG. 5C). In contrast, the Ku70 mutant expressing only the C-terminal 74 amino acids of Ku70 (Ku70 (536-609)), which does not have Ku80-binding domains, did not rescue Bax-deficient cells from UVC-irradiation-induced cell death (FIG. 5C). This mutant could attenuate STS- and UVC-induced apoptosis in Bax-expressing cells (HEK293T and HeLa cells) (FIGS. 1 and 2). Furthermore, down regulation of Ku70 did not induce hypersensitivity to STS in Bax-deficient cells (FIG. 5D), although the reduced Ku70 level by antisense Ku70 RNA in Bax-deficient cells was low enough to enhance Bax-overexpression-induced apoptosis (FIG. 5D). These results suggest that the anti-apoptotic activity of Ku70 depends on the expression of Bax. In addition, Ku70 overexpression did not suppress apoptosis induced by the “extrinsic” cell death stimuli, such as Fas and TRAIL in HeLa cells, which can induce apoptosis without mitochondrial Cytochrome c release (Ashkenazi and Dixit, 1998) (FIGS. 5E and F). As shown in FIG. 1B, Ku70 could not inhibit Bak-overexpression induced cell death. Taken together, these results support the hypothesis that Ku70 suppresses cell death by inhibiting Bax-mediated apoptotic signals.

Ku70 Inhibits the Mitochondrial Translocation of Bax

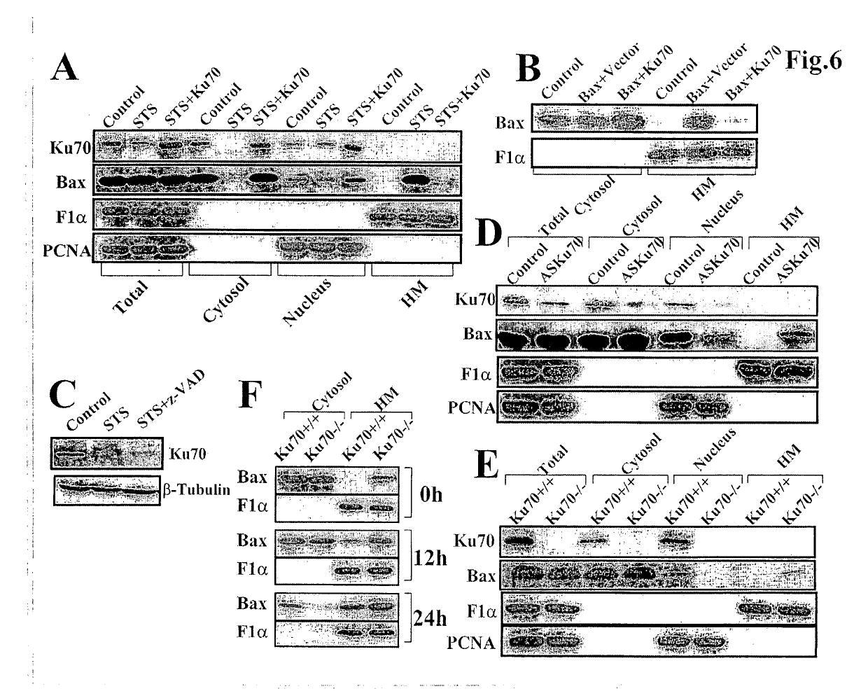

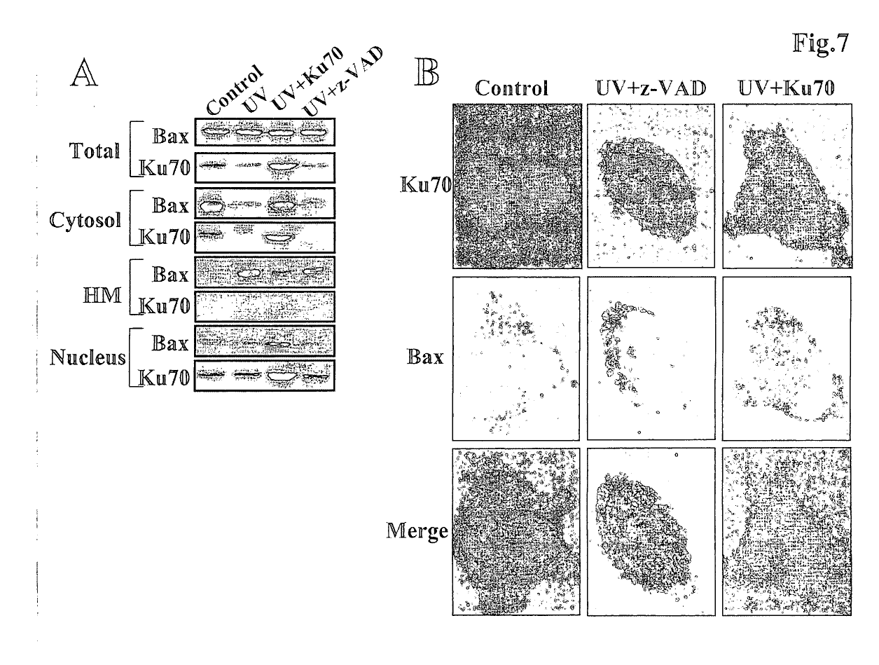

Next, we examined the subcellular localization of Bax and Ku70 during apoptosis (FIGS. 6 and 7). As previously reported, Bax translocates from the cytosol to mitochondria (Heavy Membrane fraction) in response to apoptotic stimuli (Wolter, et al., 1997), and Bax was also detected in the nucleus (Hoetelmans, et al., 2000; Mandal, et al., 1998; Nishita, et al., 1998; Salah-eldin, et al., 2000). Ku70 suppressed the mitochondrial translocation of Bax induced by STS-treatment (FIG. 6A) and UVC-irradiation (FIG. 7). On the other hand, Ku70 was detected both in the cytosol and the nucleus under normal conditions as reported (Fewell and Kuff, 1996), but not in mitochondria-enriched fraction (Heavy Membrane (HM) fraction) (FIGS. 6 and 7). The elevation of Ku70 levels also suppressed the accumulation of Bax in mitochondrial fraction caused by overexpression of Bax proteins whereas it increased the relative proportion of Bax in the cytosol (FIG. 6B). These results suggest that Ku70 has a role in sequestering Bax from mitochondria, thus protecting cells from death.

Ku70 levels decrease significantly during apoptosis in Western blot analysis (FIG. 6A). Importantly, only cytosolic Ku70 levels decreased, and nuclear Ku70 levels remained constant during apoptosis (FIGS. 6A and 7A) as previously observed (Yang, et al., 2000). This change was not affected by Caspase inhibitor treatments (FIGS. 6C and 7A). Fragmented Ku70 with a smaller molecular weight was not detected during STS- and UVC-irradiation-induced apoptosis (Supplemental data H). The disappearance of immunoreactive Ku70 on the Western blot may be due to the proteolysis or the post-translational modification of Ku70 causing the loss of immunoreactivity of this protein. Since Ku70 binds and inhibits the mitochondrial translocation of Bax, the disappearance of immunoreactive-Ku70 in the cytosol fraction may be one of the early caspase-independent events in apoptosis that causes the dissociation of Ku70 from Bax.

Consistent with the hypothesis that Ku70 is a cytosol retention factor of Bax, increased Bax protein association with mitochondria was observed both in Ku70-antisense RNA-expressed cells and in Ku70−/−MEFs (FIGS. 6D and E). Furthermore, mitochondrial translocation of Bax during STS-induced apoptosis occurred earlier in Ku70−/−MEFs (FIG. 6F). However, the dissociation of Ku70 from Bax may not be sufficient for the apoptotic level of Bax accumulation in mitochondria. Although lowering Ku70 levels itself increased Bax association with mitochondria, substantial amount of Bax remained in the cytosol under these conditions (FIGS. 6D and E).

Bax levels in the nucleus was increased by Ku70-overexpression (FIGS. 6A and 7A), but was decreased by the down regulation of Ku70 (FIG. 6D). In addition, Ku70-deficient cells did not show detectable Bax in the nuclear fraction (FIG. 6E). These results suggest that Ku70 is required for the nuclear localization of Bax. Since Ku70 and Bax interact with each other, Ku70's nuclear localization signal (Koike, et al., 2001) may play roles for the nuclear transport of Bax.

### Discussion

Ku70 has been recognized as a subunit of Ku-protein complex comprised of two subunits (Ku70 and Ku80) that plays an important role in non-homologous DNA double-strand brake repair (Khanna and Jackson, 2001; Walker, et al., 2001). The heterodimerization of Ku70 and Ku80 is a prerequisite for DNA end-joining activity (Khanna and Jackson, 2001; Walker, et al., 2001). It has been reported that Ku80binding domains on Ku70 (609 amino acids) are localized in amino acids of 1-115 and 430-482 (Wang, et al., 1998). The present study showed that the C-terminal 74 amino acids of Ku70, which do not have Ku80-binding domains, are sufficient for the inhibition of Bax-mediated apoptosis (FIGS. 1 and 2). These results suggest that Ku70 has the Bax-inhibitor activity independent from the previously recognized DNA-damage repair function. Consistent with this hypothesis, it has been reported that fibroblasts derived from Ku70-deficient mice become hypersensitive to several anti-cancer drugs that does not induce DNA-damage (Kim, et al., 1999). We also observed that Ku70-deficient cells showed increased sensitivity to STS-treatment that induces apoptosis regardless of DNA-damage (FIG. 3). This phenotype of Ku70-deficient cells may be explained by the anti-Bax activity of Ku70. In addition, increased neuronal cell death in the developing brain of Ku70-deficient mice may be partly explained by the abnormal activation of Bax due to the absence of Ku70, since Bax plays a key role in neuronal apoptosis during the development (Deckwerth, et al., 1996; Kim, et al., 1999).

Previously, the presence of “cytosol retention” signal in the N-terminus of Bax has been suggested by in vitro experiments (Goping, et al., 1998), which is consistent with our observation that N-terminus of Bax is required for Ku70 to inhibit the mitorchondrial localization of Bax (FIGS. 4 and 6). Although the present data suggest that the N-terminal 53 amino acids of Bax is required for Ku70 binding, it is not yet clear whether this N-terminus region is the binding domain of Ku70, since the binding of N-terminal portion of Bax and Ku70 could not be examined due to the difficulty of the expression of this peptide. The possibility remains that Ku70 binds to the portion other than N-terminus of Bax. The deletion of N-terminal amino acids may modify the conformation required for the interaction with Ku70, and therefore Ku70 may not be able to bind to Bax. At present, our data with a series of Bax-deletion mutants suggest that C-terminal transmembrane domain, 2^{nd} α-helix (BH3 domain) (FIG. 4), and putative channel formation domain (α-helix 5 and 6) (not shown) are not required for Ku70-binding to Bax. Further biochemical analysis of the interaction of Bax and Ku70 using purified protein will be required to examine the character of the interaction.

The present data suggest that cytosolic Ku70 has an activity to interfere with the mitochondrial translocation of Bax (FIGS. 6 and 7). However, the absence of Ku70 was not sufficient for the apoptotic level of Bax translocation from the cytosol to mitochondria, when there is no apoptotic stresses (FIGS. 6D and E). These results suggest the presence of other factors activated by apoptotic stimuli that enhance Bax relocation. One possibility is that Ku70 may not be the only cytosol retention factor of Bax. Dissociation of multiple cytosol retention factors from Bax may be required for the complete relocation of Bax from the cytosol to mitochondria. Another possibility is the presence of the factors that are actively trafficking Bax into the mitochondrial membrane. In this regard, the C-terminal ninth α-helix of Bax, which is not required for Ku70/Bax interaction, has been reported to play a role in mitochondrial targeting (Suzuki, et al., 2000). The combination of factors regulating cytosol retention with the N-terminus and the mitochondrial targeting of the C-terminus of Bax may set the threshold for apoptosis induction through Bax. In addition to this system, BH3 domain proteins such as Bid will also play critical roles, for example, in activation of Bax after its translocation to mitochondria (Wei, et al., 2001).

Bax levels in the nucleus were increased by Ku70-overexpression (FIGS. 6A and 7A), and were significantly reduced by lowering Ku70 levels (FIGS. 6D and E). These results suggest that Ku70 is required for the nuclear localization of Bax. Ku70/Bax complex may be targeted to the nucleus via Ku70's nuclear localization signal. Although most of the studies about the subcellular localization of Bax focus on the partitioning of Bax in the cytosol and mitochondria, it has been known that Bax also exists in the nucleus (Hoetelmans, et al., 2000; Mandal, et al., 1998; Nishita, et al., 1998; Salaheldin, et al., 2000). However, the biological activity of Bax in the nucleus is not understood. From the viewpoint of Ku70's DNA-repair function, Bax may be an inhibitor of Ku70 when it resides in the nucleus. The C-terminal portion of Ku70, where Bax binds, is also reported to be a target of radiation-induced proapoptotic protein (called Clusterine, XIP8, TRPM-2, or SGP-2) (Yang, et al., 2000). Bax may co-operate together with these factors to suppress DNA-damage repair in the nucleus.

Cytosolic Ku70 levels decreased significantly during apoptosis when Ku70 levels were examined by Western blot (FIGS. 6A, C, and 7A) and immunohistochemistry (FIG. 7B). This change was not affected by caspase-inhibitor. These results imply that caspase-independent Ku70 proteolysis occurs in the early phase of apoptosis. Since Ku70 suppresses the mitochondrial translocation of Bax, dissociation of Ku70 from Bax may be one of the critical steps in the activation process of Bax. However, the decrease of Ku70 levels in Western blot and immunohistochemistry does not necessarily indicate the proteolysis of this protein. For instance, the post-translational modification of Ku70 that abolishes the immunoreactivity of Ku70 protein may be the reason for the disappearance of Ku70. Decrease of Ku70 levels in Western blot analysis was detected only in the cytosol but not in the nuclear fraction, suggesting that putative Ku70-protease(s) or -modifier(s) exist in the cytosol but not in the nucleus. Caspase-independent proteolytic pathways have been implicated to play roles in apoptosis, such as ubiquitin/proteosome- and calpain-mediated proteolysis (Johnson, 2000), and one of these proteolytic mechanisms may be involved in the mechanism of Ku70 disappearance during apoptosis.

In summary, we found that Ku70 interacts with Bax, and inhibits mitochondrial translocation of Bax. We also found that nuclear localization of Bax requires Ku70. Our data suggest that Ku70 has a physiological role in the regulation of apoptosis in addition to the previously known roles in DNA-damage repair. Several anti-cancer drugs are known to stimulate Bax-mediated apoptotic signals. Irregular high expression levels of Ku70 in cancer cells have been reported (Wilson, et al., 2000; Zhao, et al., 2000). The elevated Ku70 levels may confer cancer cells resistance to anti-cancer drugs triggering Bax-mediated apoptosis. On the other hand, rapid reduction of Ku70 levels occurs in the early phase of ischemia-induced tissue damage (Kim, et al., 2001). This Ku70 proteolysis may enhance Bax-mediated cell death in the damaged tissue by ischemia. The regulation of Ku70 levels in the cells may alter the sensitivity of the cells to the stresses that trigger intrinsic cell death signals.

### Experimental Procedure

Plasmid

The plasmids pGilda-Bax, pcDNA3-Bax (human), pcDNA3-Myc-XIAP, pcDNA3-Bcl-2 (human), and pcDNA3-BcIXL (human) have been described (Deveraux, et al., 1997; Matsuyama, et al., 1998b). Yeast expression plasmid libraries of cDNAs from HeLa cells (pJG4-5 vector, In Vitrogen) and mouse brain (PYES vector, In Vitrogen) were constructed using directional cDNA synthesis kit (Stratagene) according to the manufacturer's manual. The plasmid vectors pCMV-2B and pEGFP were purchased from Stratagene and Clontech, respectively, and human full length of Ku70 and the deletion mutants of Ku70 were subcloned into BamH1 and Sal1 sites of pCMV-2B vector, and the deletion mutants of Bax were subcloned into EcoR1 and Xho1 sites of PEGFP plasmid. The full length Ku70 cDNA was prepared by RT-PCR using HeLa cell cDNA. The mutant constructs of Ku70 and Bax described in this article were prepared by 2^{nd} step PCR mutagenesis method (Matsuyama, et al., 1998a).

Yeast Methods

Yeast strain (EGY48) used for this study has been described previously (Matsuyama, et al., 1998b). Yeast-based functional screening of Bax-supressors was performed using pGilda-Bax as the Bax-expression plasmid according to the previously described method (Xu, et al., 2000; Xu and Reed, 1998).

Cell Culture and Apoptosis Detection

HEK293T cells, HeLa cells, and mouse embryonic fibroblasts (MEF) were cultured in DMEM supplemented with 10% fetal bovine serum (FBS). Transfection of the plasmids was performed by SUPERFECT (Quiagen) according to the manufacturer's manual. Apoptosis was induced by pcDNA3-human Bax (Bax-encoding plasmid)-transfection, Staurosporin (STS)-treatment, UVC-irradiation, anti-Fas-antibody-treatment (clone CH11), and human recombinant TRAIL-treatment (BD-Pharmingen). The amount of the plasmids, the concentration of STS, Fas-antibody, and TRAIL, and the energy of UVC-irradiation are as described in the figure legends. Apoptosis in the transfected cells were analyzed as follows: Plasmid encoding EGFP (0.5 ug of pEGFP) was transfected to all the groups to mark the transfected cells. One day following transfection of the plasmids listed in the figure legends or treatment of the cells with staurosporin (STS) or UVC-irradiation, cells were stained with Hoechst dye and cells with apoptotic nuclei were counted in GFP expressing cells under fluorescent microscope as previously reported (Wei, et al., 2001). Each point in the figures showing percentages of apoptosis represents the mean±SE of three experiments. Caspase activities of cells were measured by detecting the cleavage of fluorogenic substrate of caspase (DEVD-afc) as previously described (Deveraux, et al., 1997).

Cytochrome c Detection

One day following the transfection of the plasmids or the treatment of the cells with STS or UVC-irradiation, cells were re-suspended in 200 ul of homogenization buffer (250 mM Sucrose, 20 mM HEPES, pH 7.5, 10 mM KCI, 1.5 mM MgCl₂, 1 mM EDTA, 0.1 mM phenylmethylsulfonyl fluoride), and separation of the cytosol and heavy membrane fraction (containing mitochondria and ER) were performed as previously reported (Goldstein, et al., 2000; Wang, et al., 1996). Cytosolic fraction of 20 ug protein and 1 ul of membrane fraction (out of total 50 ul) were analyzed by Western-blot with Cytochrome c antibody (BD-Pharmingen dilution 1:1000).

Immunoprecipitation

Co-immunoprecipitation of endogenous Ku70 and Bax: 10⁷ cells of HEK293T cells were lysed in 200 ul of “Chaps-based buffer” (150 mM NaCI, 10 mM Hepes, pH7.4, 1% Chaps) or “detergent-free hypotonic buffer” (hypotonic (5 mM NaCl) phosphate buffered saline, pH 7.4) containing protease inhibitors (100 times dilution of Protease Inhibitors Cocktail; SIGMA) according to the previously reported method (Hsu and Youle, 1998). For the experiments in “detergent free” condition, the cytosol fraction was used and NaCI was added to prepare the isotonic condition before immunoprecipitation as previously reported (Hsu and Youle, 1998). Immunoprecipitation was performed as follows according to the previous methods (Hsu and Youle, 1998, Matsuyama, 1998a). After precleaning of 600 ul of the sample with 50 ul of Protein G-Sepharose at 4° C. for 1 hour, immunoprecipitations were performed by incubating 200 ul of lysates with 20 ul of Protein G-Sepharose preabsorbed with 2 ug of anti-Bax polyclonal antibody or 2 ug of anti-Ku70 monoclonal antibody at 4° C. for 2 hours. After extensive washing in the buffer, beads were boiled in 40 ul of Laemmli buffer and 20 ul of the eluted proteins were subjected to SDS-PAGE immunoblot analysis. Normal rabbit serum (NRS) and mouse IgG were used as negative controls. Western Blot analysis of pre-immunoprecipitation (20 ug protein) (Input) and immunoprecipitated samples (IP) were performed by anti-Ku70 monoclonal antibody (BD-Pharmingen) or anti-Bax polyclonal antibody (BD-Pharmingen). Co-immunoprecipitation of GFP-Bax and Ku70: Co-immunoprecipitation of GFP-Bax and Ku70. HEK293T cells (10⁶ cells) were transfected with 1.0 ug pEGFP (GFP), pEGFP-Bax (Bax), pEGFP-BaxΔN (BaxΔN), pEGFP-BaxΔα2 (BaxΔα2), or pEGFP-BaxΔα9 (BaxΔα9) in the presence of 50 uM z-VAD-fmk. One day following transfection, cells were collected in Chaps-based buffer and co-immunoprecipitation experiments of GFP-Bax and endogenous Ku70 were performed. Anti-GFP polyclonal antibody (2 ug for 200 ul sample) (Invitrogen) for immunoprecipitation (12% SDS-PAGE), and anti-Ku70 monoclonal antibody (BD-Pharmingen) for the detection of Ku70 (10% SDS-PAGE). BaxΔN (deletion of amino acids 1-53), BaxΔα2 (deletion of amino acids 33-71), and BaxΔα9 (deletion of amino acids 170-192) were prepared using 2^{nd} step PCR-mutagenesis methods as reported (Matsuyama, et al., 1998a). Co-immunoprecipitation of Flag-tagged-Ku70 and endogenous Bax: HEK293T cells (10⁶ cells) were co-transfected with 1.0 ug pcDNA3-Bax and 1.0 ug pCMV-2B-control vector (Flag-tagged firefly luciferase), pCMV-2B-Ku70 wt (Flag-Ku70 wt), pCMV-2B-Ku70₁₋₅₃₅(Flag-Ku70₁₋₅₃₅), pCMV-2B-Ku70₄₉₆₋₆₀₉(Flag-Ku70₄₉₆₋₆₀₉) or pCMV-2B-Ku70₅₃₆₋₆₀₉(Flag-Ku70₅₃₆₋₆₀₉) in the presence of 50 uM z-VAD-fmk. Co-immunoprecipitation was performed with anti-Flag monoclonal antibody (2 ug for 200 ul sample), and Western-blot of Bax (15% SDS-PAGE) was done with anti-human Bax polyclonal antibody (BD-Phramingen).

Subcellular Fractionation

One day after the treatment, cells were homogenized (Teflon homogenizer) with 200 ul of ice-cold homogenization buffer (250 mM Sucrose, 20 mM HEPES, pH 7.5, 10 mM KCI, 1.5 mM MgCI₂, 1 mM EDTA, 0.1 mM phenylmethylsulfonyl fluoride). Subcellular fractionation was performed as reported (Hoetelmans, et al., 2000), together with the confirmation of each fraction with appropriate marker proteins (nucleus fraction; PCNA by anti-human PCNA antibody (Oncogene), mitochondria containing heavy membrane fraction; F1-ATPase α-subunit by anti-F1α subunit antibody (Molecular Probe). For total cell lysates, samples were prepared with ice-cold lysis buffer (containing 50 mM NaCl, 25 mM Hepes (pH 7.4), 1 mM EDTA, 1 mM EGTA, 1 mM PMSF, 10 ug/ul E-64, and 1% Triton X-100). In the experiments of FIGS. 6A, B, D-F, and 7A, samples containing 20 ug of protein from total cell lysates or cytosol fraction were subjected to Western-blot analysis of Ku70 and Bax. The pellets of the fractions of heavy membrane and nuclear were dissolved in 50 ul of SDS-PAGE sample buffer, and the same proportion of the volume equal to that of the cytosol samples out of its total volume was used for Western-blot analysis.

Confocal Microcope Image

HEK293T cells (10⁶ cells) were transfected with 1.0 μg pCMV-2B (Control and UV+z-VAD) or pCMV-2B-Ku70 (UV+Ku70). One day following transfection, except in the control group (Control), cells were exposed to UVC-irradiation in the absence (UV+Ku70) or the presence of 50 μM z-VAD-fmk (UV+z-VAD). Twelve hours after UVC-irradiation (200 J/m²), cells were fixed with 4% paraformaldehyde in PBS for 10 minutes. Aspirate 4% paraformaldehyde and wash cells twice with PBS. Cells were permeablized with 0.5% Triton X-100 in PBS for 5 minutes and then incubated in 0.05% Tween-20 in PBS for 5 minutes at room temperature. Fixed cells were pre-incubated for 30 minutes in PBS containing 5% BSA at 37° C. before immunostaining. Cells were double stained with anti-Bax-monoclonal antibody (Pharmingen, dilution 1:50) and anti-Ku70-polyclonal rabbit antibody (Santa-Cruz, dilution 1:50) following with the detection of FITC-labeled anti-rabbit IgG (Jackson ImmunoResearch, dilution 1:100) and TexasRed-labeled anti-mouse IgG (Jackson ImmunoResearch, dilution 1:100). Microscopic analysis was performed by the confocal microscope (BioRad).

II. Antisense Ku70 RNA Increased the Efficiency of Bax-Stimulating Anti-Cancer Drugs.

We found that Ku70 has a new physiological function as a Bax inhibitor. Since Ku70 is an inhibitor of Bax, the reduction of Ku70 levels by antisense Ku70 sensitized HeLa cell and 293 cells to Bax-mediated cell death, as shown in FIG. 3. The commonly used anti-cancer drugs such as ETOPOSIDE, CISPLATIN, and DOXORUBICIN are known to induce cell death in cancer cells by activating Bax-mediated cell suicide pathway (Reed, 1996; Reed, 1998; Zhang, et al., 2000). Therefore, the reduction of Ku70 levels in cancer cells are expected to improve the efficiency of these anti-cancer drugs to eliminate the malignant cells. The following data show the evidences that the reduction of Ku70 actually increases the sensitivity of several types of cancer cells to the anti-cancer drugs.

Our data show that the effectiveness of antisense Ku70 RNA depends on the expression levels of Ku70 and Bax in the cells. In this study, antisense Ku70 RNA was expressed by plasmid transfection that encodes the reversed Ku70 CDNA. The expression levels of Bax and Ku70 in HeLa cells are used as the standard levels to diagnose the levels of these proteins in other cancer cells, because HeLa cells are the first human cell line and have been a commonly used model cell in molecular biology.

Antisense Ku70 RNA significantly increased the efficiency of Bax-stimulating anti-cancer drugs to eliminate cancer cells in the cells expressing standard levels of Bax and Ku70. However, antisense Ku70 has no or less effects in cells with no or less expression of Bax, respectively, because the anti-apoptotic role of Ku70 comes from the inhibition of Bax.

On the other hand, Ku70 levels in cancer cells are also important factor. Antisense Ku70 show less effect in inducing hypersensitivities to anti-cancer drugs in cancer cells with a low level of Ku70 because Bax in these cells is already almost free from Ku70's inhibition. These observations suggest that the examination of the levels of Ku70 and Bax in cancer cells can predict the effectiveness of antisense Ku70 to increase the efficiency of cancer cell killing by Bax-stimulating anti-cancer drugs.

FIGS. 8A and B show the protein expression levels of Bax and Ku70 in glioma cells (U87-MG, T98-G, U373-MG, U251-MG, SNB-19, and A-172), HeLa cells, hepatoma (Hep3B), colon cancer cells (HCT-116), fibrosarcoma cells (HT-1080), prostate cancer cells (LNCaP and Du145), and breast cancer cells (MCF-7 and MDA-MB-468). Since HeLa cell is the first human cell line and has been the most commonly used model cell in molecular biology, the expression levels of Bax and Ku70 in HeLa cells are used as the standard levels to diagnose the levels of these proteins in other cancer cells. Although most of the cell lines (nine out of thirteen) examined in FIG. 8 expressed standard levels of Bax and Ku70, two glioma cells (U373-MG and A-172), one colon cancer cells (HCT-116), and one prostate cancer (Du145) show different phenotypes. Ku70 levels are low in two glioma cells (U373-MG and A-172), and Bax levels are low in one colon cancer cells (HCT-116) and one prostate cancer cells (Du145).

FIG. 8C demonstrates that antisense Ku70 RNA down-regulates Ku70 level specifically in the cells without non-specific effects on the levels of other proteins regulating apoptosis such as Bax, Bcl-2, and Bcl-X.

Bax is a cell death-inducing protein. However, it resides in the cytosol as a quiescent protein in the normal condition. Upon the apoptotic stimuli, Bax translocates into mitochondria and stimulates mitochondria to release apoptogenic factors to induce cell suicide. We found that Ku70 binds Bax in the cytosol and prevents its mitochondrial translocation. Therefore, the reduction of Ku70 by antisense Ku70 can enhance the mitochondrial translocation of Bax in the cells stimulated by apoptotic stimuli including Bax-activation anti-cancer drugs.

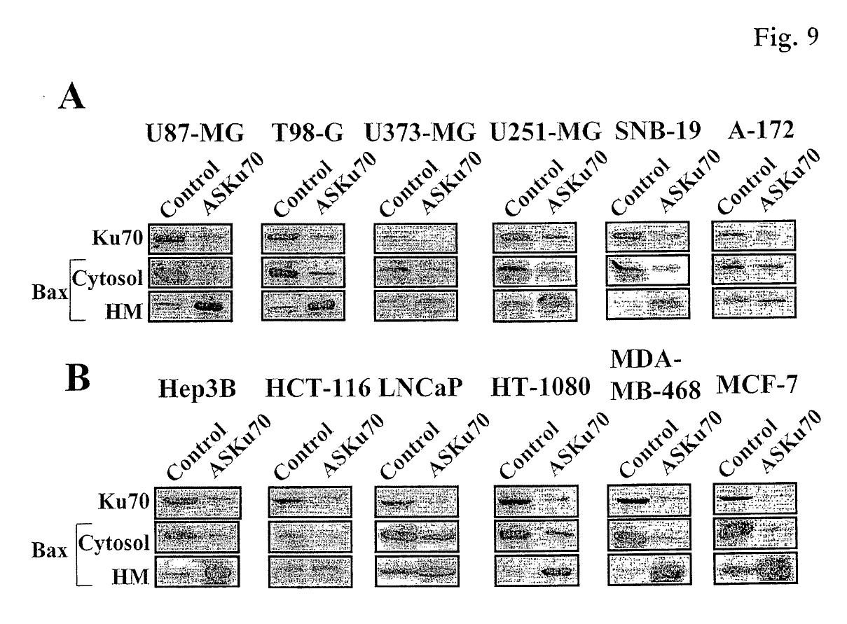

FIG. 9 shows that the reduction of Ku70 levels by antisense Ku70 enhances the mitochondrial translocation of Bax stimulated by etoposide, one of the commonly used anti-cancer drugs. The plasmids encoding antisense Ku70 or the vector control were transfected to the cells, and then cells were treated by Bax-stimulating anti-cancer drug, ETOPOSIDE. One day after ETOPOSIDE treatment, cells were collected and subcellular fractionation was performed, and Bax levels in the fractions of the cytosol and mitochondria (heavy membrane: HM in the figure) were determined by Western blotting. In most types of cancer cells examined, antisense Ku70 treatment enhanced the translocation of Bax from the cytosol fraction to the mitochondria fraction. However, the cancer cell line with low levels of Ku70 (two glioma cell lines: U373-MG and A-172) or Bax shows (one colon cancer cell line: HCT-116) smaller difference between control and antisense Ku70 treated group.

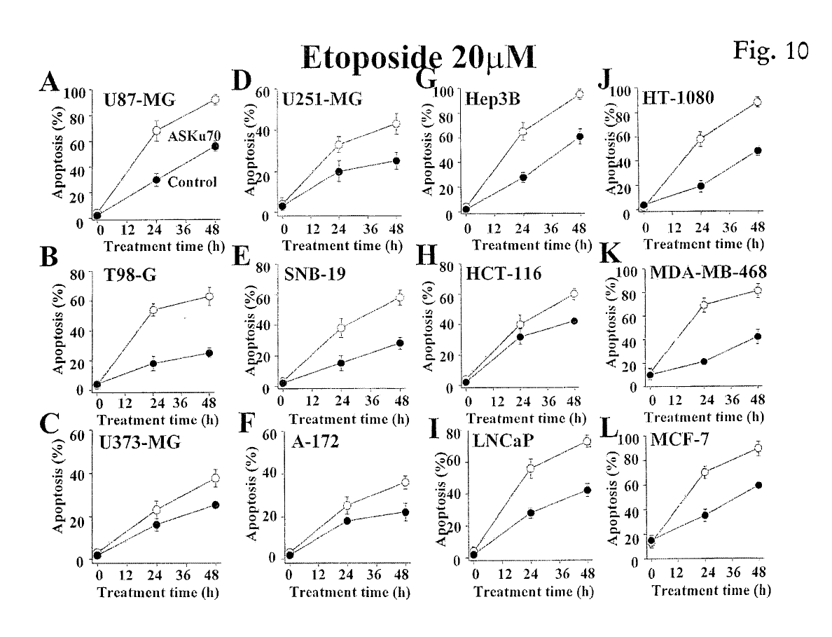

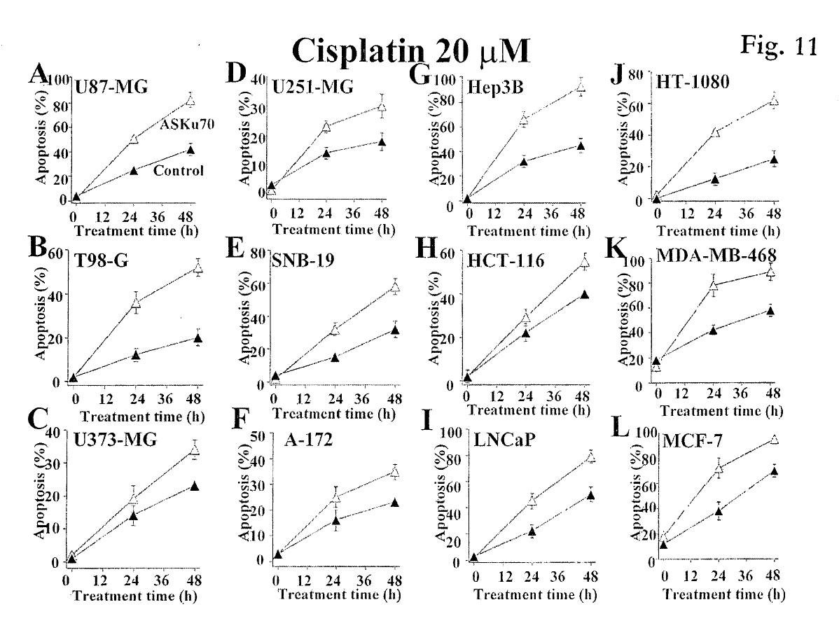

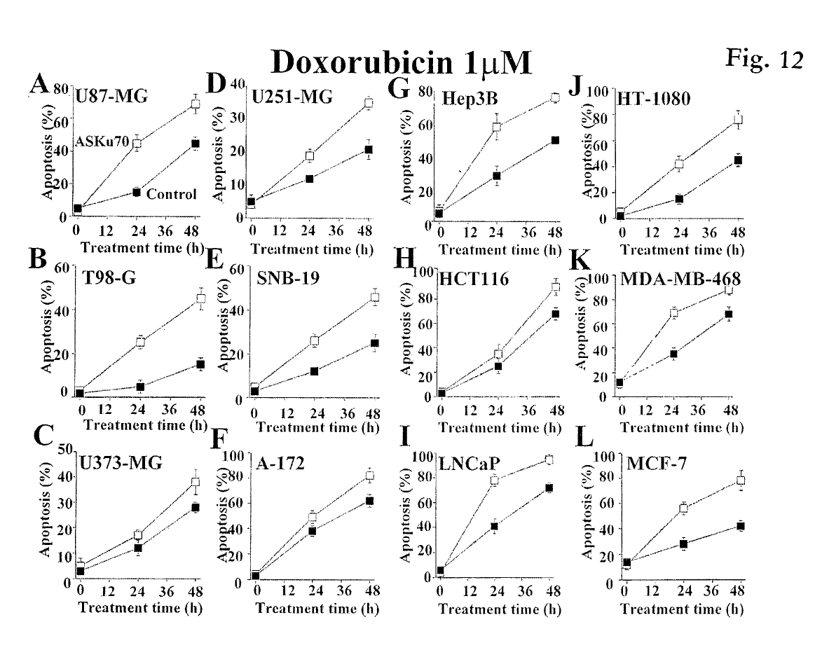

The effects of antisense Ku70 to increase the efficiency of anti-cancer drugs to kill cancer cells are shown in FIGS. 10-13. Twelve cancer cell lines are transfected with the plasmid encoding antisense Ku70 or the vector control. One day following the transfection, cells were treated by three Bax-stimulating anti-cancer drugs; ETOPOSIDE (20 uM) (FIG. 10), CISPLATIN (20 uM) (FIG. 11), or DOXORUBICIN (1 uM) (FIG. 12). The percentages of apoptotic cells were measured at 24 and 48 hours after the addition of anti-cancer drugs in the medium (FIGS. 10-12). Antisense Ku70 treatment showed significant increase of the killing activity of the cancer cells by these anti-cancer drugs in nine cancer cell lines (FIGS. 10-12). These nine cancer cell lines are A: glioma cell line U87-MG, B: glioma cell line T98-G, D: glioma cell line: U261-MG, E: glioma celiline SNB-19, G: hepatoma cell line Hep3B, I: prostate cancer cell line LNCaP, J: fibrosarcome cell line HT-1080, K: Breast cancer cell line MDA-MB-468, and L: breast cancer cell line MCF-7. However, antisense Ku70 treatment induced only slight effects to increase the killing efficiency of anti cancer drugs in three cancer cell lines that has low levels of Ku70 or Bax (see FIG. 8 also for Ku70 and Bax levels). These cell lines are C: glioma cell line U373-MG, F: glioma cell line A-172, and H: colon cancer cell line HCT-116. These results suggest that the levels of Ku70 and Bax can be the diagnostic markers to predict the effectiveness of antisense Ku70 to increase the efficiency of anti-cancer drugs to eliminate cancer cells.

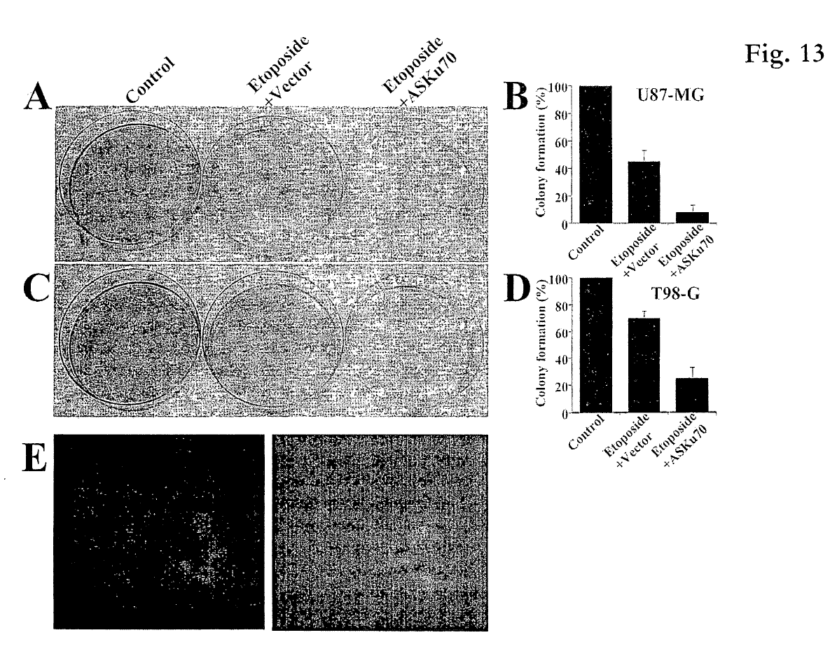

FIG. 13 shows the effects of antisense Ku70 in increasing the activity of etoposide to suppress cancer cell growth in the long term culture (three weeks). Two glioma cell lines (U87-MG and T98-G) were transfected with the plasmid encoding antisense Ku70 or the vector control. One day after the transfection, 20 uM etoposide was added to the culture and the growth activity (cell dividing activity) was examined by measuring the number of the colonies formed on the plates during three weeks culture after etoposide addition to the culture. FIGS. 13A and C showed the picture of the colonies on the plates stained with hematoxylane. FIGS. 13B and D shows the relative number of the colonies formed in the etoposide-treated cells transfected with vector control plasmid (ETOPOSIDE+vector) and antisense Ku70 encoding plasmid (ETOPOSIDE+AS Ku70) of Antisense-Ku70 treatment significantly enhanced the suppression of the cancer cell growth by etoposide in two glioma cell lines (A and B: U87-MG, C and D: T98-G). These results are consistent with the observations that antisense Ku70 treatment increase the efficiency of cancer cell killing by anti-cancer drugs in two days culture (FIGS. 10-12).

FIG. 13E shows the example of apoptotic cells in antisense Ku70 treated cells. A glioma cell line (T98G) was transfected with the plasmids encoding antisense Ku70 and Green Fluorescent Protein (GFP). The transfected cells (i.e. antisense Ku70 RNA expressing cells) can be detected by green fluorescence under the microscope (FIG. 13E right panel). One day after the following the plasmid transfection, cells were treated by ETOPOSIDE (20 uM) for 24 hours and the nuclei of the cells were stained by Hochst-dye (FIG. 13E left panel). The cells expressing antisense Ku70 RNA (green cells in the right panel) show typical apoptotic nuclei (nuclear fragmentation) (right panel).

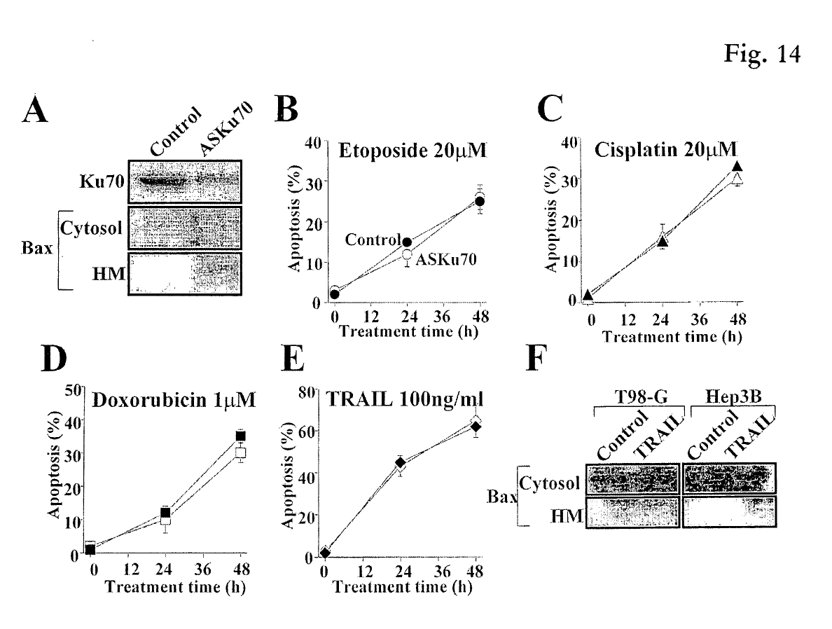

Since Ku70 suppresses apoptosis by inhibiting Bax activity, the regulation of Ku70 levels does not change the sensitivity of Bax-deficient cells to the apoptosis-inducing anti-cancer drugs. FIG. 14 shows the evidences that antisense Ku70 treatment does not induce hypersensitivities of Bax-deficient cells (prostate cancer cell Du145, see also FIG. 10 for the expression level of Bax) to anti-cancer drugs. Antisense Ku70 expression reduced Ku70 levels in Du145 (FIG. 14A upper lane). Bax could not be detected the fractions of the cytosol and mitochondria as reported (Rampino, et al., 1997) (FIG. 14A middle and lower lanes).

Bax-deficiency in Du145 is known to be due to the frame shift mutation in the promoter region of Bax gene in the chromosome (Rampino, et al., 1997). In this Bax-deficient prostate cancer cells, antisense Ku70 treatment did not increase the cell killing activity by ETOPOSIDE (20 uM) (FIG. 14B), CISPLATIN (20 uM) (FIG. 14C), and DOXORUBICIN (1 uM) (FIG. 14D). These results suggest that the levels of Bax in the cells can be a diagnostic marker to predict the effectiveness of antisense Ku70 to induce hypersensitivities of cancer cells to anti-cancer drugs. The anti-cancer drugs examined (ETOPOSIDE, CISPLATIN, and DOXORUBICIN) are known to induce DNA-replication failure in the cancer cells that trigger mitochondria-dependent apoptosis pathway. In mitochondria-dependent cell death pathway, two cell death-inducing proteins, Bax and Bak, play a key role (Wei, et al., 2001). In Bax-deficient cells, Bak is known to stimulate mitochondria-dependent apoptosis pathway (Wei, et al., 2001). Therefore, the anti-cancer drugs examined in Bax-deficient prostate cancer cells (Du145, FIG. 14) probably kill the cells by activating Bak. Since Ku70 does not inhibit Bak (FIG. 1), the reduction of Ku70 levels by antisense Ku70 could not increase the effectiveness of these drugs to kill cancer cells.

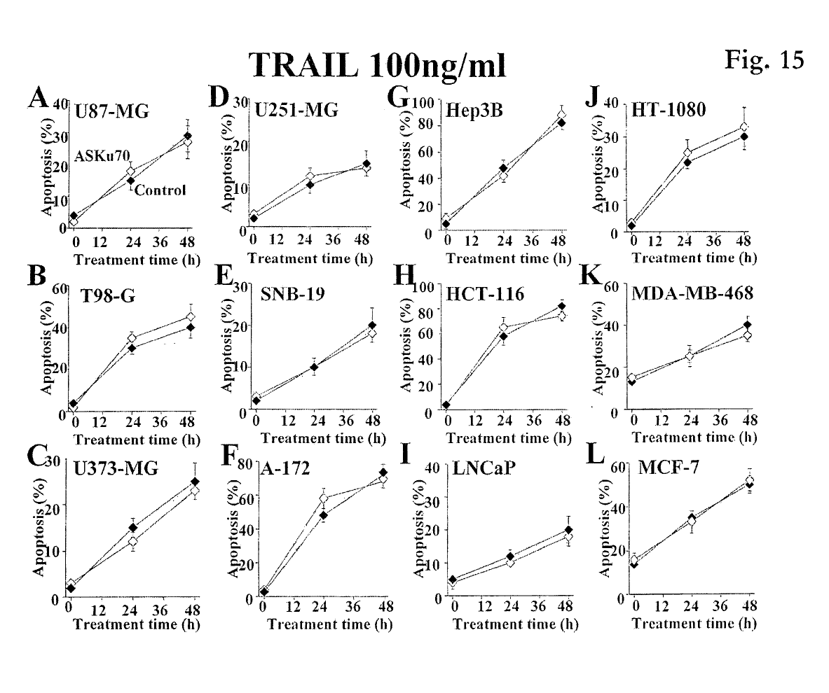

Recently, a cytokine named TRAIL (Tumor Necrosis Factor-Related Apoptosis-Inducing Ligand) was reported to show cancer cell killing activity (Gura, 1997). There are two major pathways in apoptosis, one is mitochondria-dependent pathway and the other is receptor-mediated pathway (Green and Reed, 1998). Bax plays role in the mitochondria-dependent pathway, and the receptor mediated pathway can induce cell death without Bax. TRAIL induces cell death mainly through receptor mediated pathway, therefore it does not stimulate Bax. In fact, we confirmed that TRAIL treatment did not induce the mitochondrial translocation of Bax in cancer cells (glioma cell line T98G and hepatoma cell line Hep3B) as shown in FIG. 14F. Therefore, enhancement of Bax activity by the reduction of Ku70 levels through antisense Ku70 RNA expression does not induce hypersensitivities of cancer cells to TRAIL (FIG. 15: all twelve cancer cell lines). Antisense Ku70 expression did not increase cell killing activity of TRAIL in Bax-deficient cell (prostate cancer Du145, FIG. 14E), neither. These results are consistent with the hypothesis that Ku70 protects cells from apoptosis by inhibiting Bax-mediated cell death pathway.

In summary, the present data suggest that the reduction of Ku70 levels in cancer cells by antisense Ku70 RNA expression is an effective method to increase the efficiency of cancer cell killing by commonly used anti-cancer drugs such as ETOPOSIDE, CISPLATIN, and DOXORUBICIN. These anti-cancer drugs are known to stimulate Bax-mediated apoptosis pathway, therefore, the lowering of Ku70 levels may be effective to increase the efficiency of other anti-cancer drugs stimulating the similar apoptosis pathway. The presented data also suggest that the evaluation of the levels of Ku70 and Bax in cancer cells may be a diagnostic markers to predict the effectiveness of antisense Ku70 (antisense RNA, antisense oligonucleotides, DNA-zyme and RNA-zyme based antisense technologies) to induce hypersensitivities of cancer cells to the anti-cancer drugs stimulating Bax-mediated cell death signals. The newly identified anti-Bax activity of Ku70 may provide the strategies to develop the methods to eliminate cancer cells.

### REFERENCES

Adams, J. M., and Cory, S. (1998). The Bcl-2 protein family: arbiters of cell survival. Science 281:1322-1326.
Ashkenazi, A., and Dixit, V. M. (1998). Death receptors: signaling and modulation. Science 281:1305-1308.
Chan, J. Y., Lerman, M. I., Prabhakar, B. S., Isozaki, O., Santisteban, P., Kuppers, R. C., Oates, E. L., Notkins, A. L. and Kohn, L. D. (1989). Cloning and characterization of a cDNA that encodes a 70 kDa novel human thyroid autoantigen. J. Biol. Chem. 264(7):3651-3654.
Cheng, E. H., Wei, M. C., Weiler, S., Flavell, R. A., Mak, T. W., Lindsten, T., and Korsmeyer, S. J. (2001). BCL-2, BCL-X(L) sequester BH3 domain-only molecules preventing BAX- and BAK-mediated mitochondrial apoptosis. Mol. Cell 8:705-711.
Deckwerth, T. L., Elliott, J. L., Knudson, C. M., Johnson, E. M., Jr., Snider, W. D., and Korsmeyer, S. J. (1996). BAX is required for neuronal death after trophic factor deprivation and during development. Neuron 17:401-411.
Deveraux, Q. L., Takahashi, R., Salvesen, G. S., and Reed, J. C. (1997). X-linked IAP is a direct inhibitor of cell-death proteases. Nature 388:300-304.
Ferri, K. F., Jacotot, E., Blanco, J., Este, J. A., Zamzami, N., Susin, S. A., Xie, Z., Brothers, G., Reed, J. C., Penninger, J. M., and Kroemer, G. (2000). Apoptosis control in syncytia induced by the HIV type 1-envelope glycoprotein complex: role of mitochondria and caspases. J. Exp. Med. 192:1081-1092.
Fewell, J. W., and Kuff, E. L. (1996). Intracellular redistribution of Ku immunoreactivity in response to cell- cell contact and growth modulating components in the medium. J. Cell Sci. 109:1937-1946.
Goldstein, J. C., Waterhouse, N. J., Juin, P., Evan, G. I., and Green, D. R. (2000). The coordinate release of cytochrome c during apoptosis is rapid, complete and kinetically invariant. Nat. Cell Biol. 2:156-162.
Goping, I. S., Gross, A., Lavoie, J. N., Nguyen, M., Jemmerson, R., Roth, K., Korsmeyer, S. J., and Shore, G. C. (1998). Regulated targeting of BAX to mitochondria. J. Cell Biol. 143:207-215.
Green, D. R. (2000). Apoptotic pathways: paper wraps stone blunts scissors. Cell 102:1-4.
Green, D. R., and Reed, J. C. (1998). Mitochondria and apoptosis. Science 281:1309-1312.
Gross, A., McDonnell, J. M., and Korsmeyer, S. J. (1999). BCL-2 family members and the mitochondria in apoptosis. Genes Dev. 13:1899-1911.
Gura, T. (1997). How TRAIL kills cancer cells, but not normal cells. Science 277:768.
Hoetelmans, R., van Slooten, H. J., Keijzer, R., Erkeland, S., van de Velde, C. J., and Dierendonck, J. H. (2000). Bcl-2 and Bax proteins are present in interphase nuclei of mammalian cells. Cell Death Differ. 7:384-392.
Hsu, Y. T., and Youle, R. J. (1998). Bax in murine thymus is a soluble monomeric protein that displays differential detergent-induced conformations. J. Biol. Chem. 273:10777-10783.
Johnson, D. E. (2000). Noncaspase proteases in apoptosis. Leukemia 14:1695-1703.
Jurgensmeier, J. M., Xie, Z., Deveraux, Q., Ellerby, L., Bredesen, D., and Reed, J. C. (1998). Bax directly induces release of cytochrome c from isolated mitochondria. Proc. Natl. Acad. Sci. USA 95:4997-5002.
Khanna, K. K., and Jackson, S. P. (2001). DNA double-strand breaks: signaling, repair and the cancer connection. Nat. Genet. 27:247-254.
Kim, G. W., Noshita, N., Sugawara, T., and Chan, P. H. (2001). Early decrease in DNA repair proteins, Ku70 and Ku86, and subsequent DNA fragmentation after transient focal cerebral ischemia in mice. Stroke 32:1401-1407.
Kim, S. H., Kim, D., Han, J. S., Jeong, C. S., Chung, B. S., Kang, C. D., and Li, G. C. (1999). Ku autoantigen affects the susceptibility to anticancer drugs. Cancer Res. 59:4012-4017.
Kluck, R. M., Bossy-Wetzel, E., Green, D. R., and Newmeyer, D. D. (1997). The release of cytochrome c from mitochondria: a primary site for Bcl-2 regulation of apoptosis. Science 275:1132-1136.
Koike, M., Shiomi, T., and Koike, A. (2001). Dimerization and nuclear localization of ku proteins. J. Biol. Chem. 276:11167-11173.
Korsmeyer, S. J., Wei, M. C., Saito, M., Weiler, S., Oh, K. J., and Schlesinger, P. H. (2000). Pro-apoptotic cascade activates BID, which oligomerizes BAK or BAX into pores that result in the release of cytochrome c. Cell Death Differ. 7:1166-1173.
Mandal, M., Adam, L., Mendelsohn, J., and Kumar, R. (1998). Nuclear targeting of Bax during apoptosis in human colorectal cancer cells. Oncogene 17:999-1007.
Matsuyama, S., Schendel, S. L., Xie, Z., and Reed, J. C. (1998a). Cytoprotection by Bcl-2 requires the pore-forming alpha5 and alpha6 helices. J. Biol. Chem. 273:30995-31001.
Matsuyama, S., Xu, Q., Velours, J., and Reed, J. C. (1998b). The Mitochondrial F0F1-ATPase proton pump is required for function of the proapoptotic protein Bax in yeast and mammalian cells. Mol. Cell 1:327-336.
Nishita, M., Inoue, S., Tsuda, M., Tateda, C., and Miyashita, T. (1998). Nuclear translocation and increased expression of Bax and disturbance in cell cycle progression without prominent apoptosis induced by hyperthermia. Exp. Cell Res. 244:357-366.
Oltvai, Z. N., Milliman, C. L., and Korsmeyer, S. J. (1993). Bcl-2 heterodimerizes in vivo with a conserved homolog, Bax, that accelerates programmed cell death. Cell 74(4):609-619.
Oltvai, Z. N., and Korsmeyer, S. J. (1994). Checkpoints of dueling dimers foil death wishes. Cell 79:189-192.
Rampino, N., Yamamoto, H., lonov, Y., Li, Y., Sawai, H., Reed, J. C., and Perucho, M. (1997). Somatic frameshift mutations in the BAX gene in colon cancers of the microsatellite mutator phenotype. Science 275:967-969.
Reed, J. C. (1996). Balancing cell life and death: bax, apoptosis, and breast cancer. J. Clin. Invest. 97:2403-2404.
Reed, J. C. (1997a). Bcl-2 family proteins: regulators of apoptosis and chemoresistance in hematologic malignancies. Semin. Hematol. 34:9-19.
Reed, J. C. (1997b). Double identity for proteins of the Bcl-2 family. Nature 387:773-776.
Reed, J. C., Jurgensmeier, J. M., and Matsuyama, S. (1998). Bcl-2 family proteins and mitochondria. Biochim. Biophys. Acta 1366:127-137.
Reed, J. C. (1998). Dysregulation of apoptosis in cancer. Cancer J. Sci. Am. 4 Suppl. 1:S8-14.
Reeves, W. H., and Sthoeger, Z. M. (1989. Molecular cloning of cDNA encoding the p70 (Ku) lupus autoantigen. J. Biol. Chem. 264(9):5047-5052.
Saito, M., Korsmeyer, S. J., and Schlesinger, P. H. (2000). BAX-dependent transport of cytochrome c reconstituted in pure liposomes. Nat. Cell Biol. 2:553-555.
Salah-eldin, A., Inoue, S., Tsuda, M., and Matsuura, A. (2000). Abnormal intracellular localization of Bax with a normal membrane anchor domain in human lung cancer cell lines. Jpn. J. Cancer Res. 91:1269-1277.
Suzuki, M., Youle, R. J., and Tjandra, N. (2000). Structure of Bax: coregulation of dimer formation and intracellular localization. Cell 103:645-654.
Walker, J. R., Corpina, R. A., and Goldberg, J. (2001). Structure of the Ku heterodimer bound to DNA and its implications for double-strand break repair. Nature 412:607-614.
Wang, H. G., Rapp, U. R., and Reed, J. C. (1996). Bcl-2 targets the protein kinase Raf-1 to mitochondria. Cell 87:629-638.
Wang, J., Dong, X., Myung, K., Hendrickson, E. A., and Reeves, W. H. (1998). Identification of two domains of the p70 Ku protein mediating dimerization with p80 and DNA binding. J. Biol. Chem. 273:842-848.
Wei, M. C., Zong, W. X., Cheng, E. H., Lindsten, T., Panoutsakopoulou, V., Ross, A. J., Roth, K. A., MacGregor, G. R., Thompson, C. B., and Korsmeyer, S. J. (2001). Proapoptotic BAX and BAK: a requisite gateway to mitochondrial dysfunction and death. Science 292:727-730.
Wilson, C. R., Davidson, S. E., Margison, G. P., Jackson, S. P., Hendry, J. H., and West, C. M. (2000). Expression of Ku70 correlates with survival in carcinoma of the cervix. Br. J. Cancer 83:1702-1706.
Wolter, K. G., Hsu, Y. T., Smith, C. L., Nechushtan, A., Xi, X. G., and Youle, R. J. (1997). Movement of Bax from the cytosol to mitochondria during apoptosis. J. Cell Biol. 139:1281-1292.
Xu, Q., Ke, N., Matsuyama, S., and Reed, J. C. (2000). Assays for studying Bax-induced lethality in the yeast Saccharomyces cerevisiae. Methods Enzymol. 322:283-296.
Xu, Q., and Reed, J. C. (1998). Bax inhibitor-1, a mammalian apoptosis suppressor identified by functional screening in yeast. Mol. Cell. 1:337-346.
Yang, C. R., Leskov, K., Hosley-Eberlein, K., Criswell, T., Pink, J. J., Kinsella, T. J., and Boothman, D. A. (2000). Nuclear clusterin/XIP8, an x-ray-induced Ku70-binding protein that signals cell death. Proc. Natl. Acad. Sci. USA 97:5907-5912.
Yang, J., Liu, X., Bhalla, K., Kim, C. N., Ibrado, A. M., Cai, J., Peng, T. I., Jones, D. P., and Wang, X. (1997). Prevention of apoptosis by Bcl-2: release of cytochrome c from mitochondria blocked. Science 275:1129-1132.
Zhang, L., Yu, J., Park, B. H., Kinzler, K. W., and Vogelstein, B. (2000). Role of BAX in the apoptotic response to anticancer agents. Science 290:989-992.
Zhao, H. J., Hosoi, Y., Miyachi, H., lshii, K., Yoshida, M., Nemoto, K., Takai, Y., Yamada, S., Suzuki, N., and Ono, T. (2000). DNA-dependent protein kinase activity correlates with Ku70 expression and radiation sensitivity in esophageal cancer cell lines. Clin. Cancer Res. 6:1073-1078.
Zou, H., Henzel, W. J., Liu, X., Lutschg, A., and Wang, X. (1997). Apaf-1, a human protein homologous to C. elegans CED-4, participates in cytochrome c-dependent activation of caspase-3. Cell 90:405-413.

We claim:

1. A method of predicting whether cancer cells would respond to therapies that are mediated through Bax-regulated apoptosis, comprising the steps of:
(a) examining intensity of the expression of a Bax gene in cancer cells relative to a control, and
(b) predicting whether the cells will respond to therapies that are mediated through Bax-regulated apoptosis, wherein a high Bax level indicates that one may lower Ku70 levels and increase sensitivity to apoptosis.

2. The method of claim 1 wherein one additionally examines intensity of expression of a Ku70 gene in the cells.

3. The method of claim 2 wherein one examines Bax and Ku70 protein levels.

4. The method of claim 2 wherein one examines Bax and Ku70 mRNA levels.

5.-16. (canceled)
